(19) United States
(12) Patent Application Publication
(10) Pub. No.: US 2015/0308939 A1
(43) Pub. Date: Oct. 29, 2015

(54) ASSESSMENT OF RISK OF HYPERTENSION AND METHODS BASED THEREON

(71) Applicant: Westfaelische Wilhelms-Universitaet, Muenster (DE)

(72) Inventor: Hans OBERLEITHNER, Muenster (DE)

(21) Appl. No.: 14/647,355

(22) PCT Filed: Nov. 26, 2013
(86) PCT No.: PCT/EP2013/074714

§ 371 Date: May 26, 2015

(30) Foreign Application Priority Data
Nov. 27, 2012 (EP) 12194442.5

(51) Int. Cl. G01N 13/04; G01N 33/49

(52) U.S. Cl. CPC G01N 13/04; G01N 33/49; G01N 2013/003

(57) ABSTRACT

Provided is a method of analysing whether the blood pressure of a subject is sensitive to sodium intake and/or of assessing the susceptibility of a subject to develop hypertension. In the method erythrocytes of the subject are suspended in two sodium chloride solutions of about physiological osmolality. The sodium chloride concentration of one of the two solutions is at least about 25 mM lower than in the other solution. After allowing the suspended erythrocytes in the two sodium chloride solutions to settle for a period of time sufficient to allow the formation of a supernatant the difference in height of the supernatant between the two sodium chloride solutions is detected. An increased difference in height of the supernatants, relative to a threshold value, indicates that the subject's blood pressure is sensitive to sodium intake and/or that the subject is susceptible to develop hypertension.

### CROSS-REFERENCE TO RELATED APPLICATIONS

The present application claims the benefit of and the priority to an application for “Assessment of Risk of Hypertension and Methods Based Thereon” filed on 27 Nov. 2012 with the European Patent Office, and there duly assigned serial number EP 12 194 442. The content of said application filed on 27 Nov. 2012 is incorporated herein by reference for all purposes in its entirety including all tables, figures, and claims—as well as including an incorporation of any element or part of the description, claims or drawings not contained herein and referred to in Rule 20.5(a) of the PCT, pursuant to Rule 4.18 of the PCT.

### FIELD OF THE INVENTION

The present invention relates to the assessment of risk of hypertension, i.e. assessing the risk of occurrence of hypertension in a subject. The invention also relates to methods based on such risk assessment. Provided is also a method of diagnosing an increased risk of developing arterial hypertension and/or of stratifying the risk of developing arterial hypertension, as well as a method of determining whether the blood pressure of a subject is sensitive to sodium intake.

### BACKGROUND OF THE INVENTION

The following description is provided to assist the understanding of the reader. None of the information provided or references cited is admitted to be prior art to the present invention.

Circulatory disorders are worldwide the number one cause of death. Their main cause is the wear-out of arterial blood vessels over the course of life. Late effects of impaired vessels are stroke (apoplexy) and cardiac infarction. Hypertension (arterial hypertension) is regarded the most important factor in the pathogenesis of cardiovascular disorders (Meneton, P, et al., Physiol Rev (2005) 85, 679-715). With more than eight billion Euro per year alone in Germany, as well as the resulting follow-up costs the treatment of hypertension is the most expensive of all diseases. Almost half of the worldwide population above 50 years of age suffers from hypertension and will, with high likelihood, suffer from the long-term effects. The main difficulty in this regard is early diagnosis.

Hypertension is diagnosed when systolic blood pressure values are consistently above 140 mm Hg and/or diastolic blood pressure values are above 90 mm Hg. It is recommended that individuals with values in the range of 120-139/80-89 be categorized as having prehypertension (Sutters, M, in: Current Medical Diagnosis and Treatment (2013), 52nd edition McGraw Hill, Ch 11). 50% of individuals with prehypertension develop hypertension within four years.

Complications associated with manifest hypertension are irreversible alterations of the vasculature and the heart as well as atherosclerosis connected to long lasting hypertension. Chronic hypertension further leads to nephrosclerosis. At the stage of irreversible alterations only “damage control” (by lifelong application of drugs, e.g., antihypertensives), can be done, but no healing as such is achievable. The onset of essential hypertension is generally at an age from 25 to 55 years. Only for about 5% of patients with hypertension a specific cause can be identified such as a drug-induced effect or a chronic kidney disease. Unfortunately only repeated measurements of the arterial blood pressure reveals existing (manifest) hypertension, so that “arterial hypertension” is only diagnosed at a stage where the blood vessels in all likelihood are already irreversibly damaged (proliferation of the connective tissue, rarefication of elastic fibers of the main arteries, hypertrophy of vascular smooth muscle).

European patent EP 2 215 480 B1 discloses that perturbation of the endothelial glycocalyx can be diagnosed in a non-invasive manner by a size distribution method as well as on the basis of glycocalyx markers, and that this analysis is useful in the diagnosis of vascular diseases.

The search for possibilities of the early diagnosis of a “potential hypertension” is of central interest, inter alia, because of its enormous economic relevance. Unfortunately early expectations that it might be possible to predict development of hypertension via a “genetic fingerprint” have not been fulfilled, as numerous studies on the human genome over the last two decades have shown (Lupton, S. J., et al., Twin Res Hum Genet (2011) 14, 295-304). Hypertension cannot be ascribed to a few dysfunctional genes or their respective gene products.

Over the past years clinical detection methods such as the method of pulse wave analysis (Gurovich A N & Braith R W, Hypertens Res (2011) 34, 166-169) or various imaging technologies (e.g. intima media thickness) have been refined to a degree that the structural/functional state of the vascular system can be conceived in detail. Apart from the relative complexity of these examinations, which does not permit a widespread use throughout the population for purely preventive purposes, these modern methods do only detect the state of the blood vessels once already visible (structural) changes have occurred. Therefore, these methods are not suitable for the early diagnosis of a disposition for hypertension.

The onset of hypertension is thought to be directly linked to intake/clearance of sodium chloride and water. The daily sodium intake in industrial countries exceeds physiological needs to a high degree. While for millions of years the evolutionary ancestors of modern man had a diet containing less than 1 g salt/day, present consumption is in the range of about 10 g salt/day. It is generally accepted that the high amount of sodium consumed via modern nutrition (rich in salt), including finished products, often overburdens the excretion capacity of the kidneys. As a result sodium is kept and stocked in the body and in the course of years blood vessels (Oberleithner, H., et al., Proc Natl Acad Sci USA (2007), 104, 16281-16286) damages to organs such as the brain, kidneys and the heart (Oberleithner, H, & de Wardener, H. E., Blood Purif (2011) 31, 82-85).

Though efforts are made worldwide to limit sodium consumption (see WHO report: http://www.who.int/dietphysicalactivity/reducingsalt/en/index.html), but this development (reduction of the everyday sodium consumption by at least 50%) will occur only very slowly, if at all. Thus the search for means of early diagnosis of a potential hypertension, i.e. of identifying an increased risk of developing hypertension, is of central interest, inter alia due to its tremendous economic importance (supra).

It would therefore be advantageous to have available means that allow the diagnosis of an increased risk of developing hypertension. It would be particularly advantageous if such means would allow carrying out a quick and easy test for a respective diagnosis.

### SUMMARY OF THE INVENTION

The present invention provides a method, a kit and a use of such kit that can be used for a quick and simple test in medical diagnosis as well as in determining whether the blood pressure of a subject is sensitive to sodium intake, in particular whether the blood pressure of a subject has an increased sensitivity to sodium intake. Arterial hypertension and related vascular diseases have high mortality rates worldwide. A major risk factors in this regard is high sodium intake. A resulting imbalance between sodium intake and renal sodium excretion results in an increased risk that the subject will develop hypertension. Sodium sensitivity, i.e., the development of hypertension in response to a sodium salt, in particular NaCl, differs among people. A method and a kit described herein can be used to determine this sodium sensitivity of an individual.

In the following a reference to a method is intended to include a kit disclosed herein, and/or the use of such kit, as applicable. Typically such a test can thereby be used to indicate whether the vascular system of an individual has an increased sensitivity to sodium intake, i.e. whether the vascular system of an individual is more sensitive to sodium intake than an average individual of the same age group. An individual with increased sensitivity to sodium intake shows an increased response in the form of rise in blood pressure. The test can be used to assess at a pre-hypertension state whether a subject will be in need of hypertension therapy and can be used in prognosis and risk assessment. Hence, precautionary measures in terms of medical prevention can be taken already at a stage where no damage has occurred in the subject's organs and circulatory system. A method or use as disclosed in this document provides quantitative data, can be performed rapidly and is easy to carry out. A method/use described herein does not require particular expert knowledge and can be carried out without cost-intensive equipment.

A method as disclosed herein may include staging, monitoring, categorizing and/or determining a subject's risk of developing hypertension and/or a hypertension induced disease, as well as staging, monitoring, categorizing and/or determination of further diagnosis and treatment regimens in a subject at risk of suffering from hypertension.

In a first aspect the present invention provides an in vitro method of analysing whether the blood pressure of a subject is sensitive to sodium intake. The method further includes detecting at least one of (i) the thickness (height) or the volume of the glycocalyx of erythrocytes of the subject, and (ii) the zeta potential of the erythrocytes, i.e. the degree of electrostatic repulsion between erythrocytes of the subject. A decreased height or volume of the glycocalyx of the erythrocytes, relative to a threshold value, indicates that the blood pressure of the subject is sensitive to sodium intake. Further, a decreased zeta potential of the erythrocytes, relative to a threshold value, indicates that the blood pressure of the subject is sensitive to sodium intake.

The method includes in some embodiments suspending erythrocytes of the subject in a solution of an inorganic salt of about physiological osmolality. Suspending the erythrocytes may in some embodiments be carried out before detecting the thickness (height) or the volume of the glycocalyx of erythrocytes of the subject, and/or detecting the zeta potential of the erythrocytes.

In some embodiments the inorganic salt is a sodium salt such as sodium chloride. In some embodiments a respective sodium salt is Na₂SO₄. In some embodiments a respective sodium salt is Na₂NO₃. In some embodiments the sodium salt is Na₂CO₃. In some embodiments the sodium salt is Na₂HPO₄ NaH₂PO₄ or Na₃PO₄. In some embodiments the inorganic salt is a potassium salt. Examples of a suitable potassium salt include, but are not limited to, KCl, Ka₂NO₃ and K₂SO₄. In some embodiments the inorganic salt is a calcium salt such as CaCl₃, Ca₃(PO₃)₂, Ca(NO₃)₂, CaCO₃ or CaCl₂. In some embodiments the inorganic salt is an aluminium salt, such as AlCl₃ or Al₂(SO₄)₃. In some embodiments the inorganic salt is a magnesium salt such as MgCl₂ or MgSO₄. In some embodiments the inorganic salt is a lithium salt. A suitable lithium salt may for example be LiCl or Li₂SO₄. In some embodiments the inorganic salt is a zinc salt such as ZnSO₄, ZnCl₂ or Zn(SO₄)₂. In some embodiments the inorganic salt is a mixture of at least two of a sodium salt, a potassium salt, a calcium salt, an aluminium salt, a lithium salt, a magnesium salt, an iron salt, a copper salt, a nickel salt and a zinc salt.

In some embodiments the height and/or the volume of the glycocalyx of erythrocytes is analysed by atomic force microscopy.

In some embodiments the zeta potential of the erythrocytes is determined using a zeta potential analyser. In some embodiments the zeta potential is analysed by comparing the aggregation tendency of erythrocytes in two different solutions, which both have an about physiological osmolality, but differ in the concentration of the inorganic salt. In such an embodiment a method according to the first aspect includes suspending erythrocytes of the subject in a first solution of an inorganic salt. The first solution is of about physiological osmolality. The method of such an embodiment also includes suspending erythrocytes of the subject in a second solution of an inorganic salt. The second solution is likewise of about physiological osmolality. In some embodiments the first and the second solution are both solutions that contain sodium chloride, which are at least essentially void of potassium chloride—herein also referred to as a sodium chloride solution. If both the first and the second solution are a sodium chloride solution, the first sodium chloride solution has a sodium chloride concentration that is lower than the sodium chloride concentration of the second solution. In one embodiment the sodium chloride concentration in the first sodium chloride solution is about 25 mM or more lower than the sodium chloride concentration of the second sodium chloride solution. In some embodiments comparing the aggregation tendency of erythrocytes in two different solutions of an inorganic salt includes allowing the erythrocytes suspended in the two solutions of an inorganic salt to settle over a period of time. In one embodiment where the inorganic salt is sodium chloride the method includes allowing the erythrocytes suspended in the first sodium chloride solution and the erythrocytes suspended in the second sodium chloride solution to settle over a period of time. The suspended erythrocytes are allowed to settle for a period of time that is sufficient to allow the formation of a supernatant. In embodiments where two different solutions of an inorganic salt are employed for analysing the aggregation tendency of erythrocytes the method further includes detecting the difference in height of the supernatant between the two solutions of an inorganic salt. In embodiments where the inorganic salt is sodium chloride the method generally includes detecting the difference in height of the supernatant between the first and the second sodium chloride solution. Generally the difference in height of the supernatant can also be detected by detecting the difference in height of the settled erythrocytes, i.e. the height of the suspension that still contains erythrocytes, between the two solutions of the inorganic salt, such as the first and in the second sodium chloride solution. An increased difference in height of the supernatant between the two solutions of the inorganic salt, e.g. between the first and the second sodium chloride solution, relative to a threshold value, indicates that the blood pressure of the subject is sensitive to sodium intake.

In some embodiments the method according to the first aspect includes comparing the height or volume of the glycocalyx of the erythrocytes to a threshold value, which may be a control measurement or a predetermined reference value. In embodiments where the inorganic salt is sodium chloride the method may include comparing the difference in height of the supernatant between the first and the second sodium chloride solution to a control measurement or to a predetermined reference value. In some embodiments the threshold value is based on the difference in height of the supernatant between a corresponding first and a corresponding second sodium chloride solution of erythrocytes of a control sample.

If a reduced height or volume of the glycocalyx of the erythrocytes, relative to a threshold value, and/or a decreased zeta potential of the erythrocytes is detected, a method according to the first aspect may include stratifying the subject for monitoring blood pressure. In embodiments where the inorganic salt is sodium chloride and an elevated difference in height of the supernatant between the first and the second sodium chloride solution has been detected, a method according to the first aspect may include stratifying the subject for monitoring blood pressure.

In some embodiments of the method according to the first aspect the aggregation tendency of erythrocytes is compared in two pairs of two different solutions, with all solutions having an about physiological osmolality. The first pair of solutions contains a first and a second sodium chloride solution. The first and the second solution of this first pair of solutions contain sodium chloride, but differ in the concentration of sodium chloride. In one embodiment the sodium chloride concentration in the first solution is about 25 mM or more lower than the sodium chloride concentration of the second sodium chloride solution of the first pair of solutions. The second pair of solutions contains a first and a second sodium solution. The first solution of this second pair of solutions contains sodium chloride. The second solution of this second pair of solutions contains an inorganic salt different from sodium chloride and is at least essentially void of sodium chloride. In one embodiment the inorganic salt included in the second solution of the second pair of solutions is a potassium salt such as potassium chloride. Erythrocytes of the subject are being suspended in the solutions of both the first and the second pair of solutions. In one embodiment the concentration of sodium chloride in the first solution of the first pair of solutions is at least essentially the same as the concentration of the different inorganic salt in the second solution of the second pair of solutions.

In some embodiments of the method according to the first aspect repeated sessions of the method according to the first aspect are carried out. Sessions of the method may be carried out at certain, e.g. predetermined, time intervals.

In a second aspect the present invention provides an in vitro method of assessing the risk of occurrence of hypertension in a subject. The method includes detecting at least one of (i) the thickness (height) or the volume of the glycocalyx of erythrocytes of the subject, and (ii) the zeta potential of the erythrocytes, i.e. the degree of electrostatic repulsion between erythrocytes of the subject. A decreased height or volume of the glycocalyx of the erythrocytes, relative to a threshold value, indicates that the blood pressure of the subject is sensitive to sodium intake. Further, a decreased zeta potential of the erythrocytes, relative to a threshold value, indicates that the blood pressure of the subject is sensitive to sodium intake.

In some embodiments the method further includes suspending erythrocytes of the subject in a solution of an inorganic salt of about physiological osmolality.

In some embodiments the inorganic salt is a sodium salt such as sodium chloride. In some embodiments a respective sodium salt is Na₂SO₄. In some embodiments a respective sodium salt is Na₂NO₃. In some embodiments the sodium salt is Na₂CO₃. In some embodiments the sodium salt is Na₂HPO₄ NaH₂PO₄ or Na₃PO₄. In some embodiments the inorganic salt is a potassium salt. Examples of a suitable potassium salt include, but are not limited to, KCl, Ka₂NO₃ and K₂SO₄. In some embodiments the inorganic salt is a calcium salt such as CaCl₃, Ca₃(PO₃)₂, Ca(NO₃)₂, CaCO₃ or CaCl₂. In some embodiments the inorganic salt is an aluminium salt, such as AlCl₃ or Al₂(SO₄)₃. In some embodiments the inorganic salt is a magnesium salt such as MgCl₂ or MgSO₄. In some embodiments the inorganic salt is a lithium salt. A suitable lithium salt may for example be LiCl or Li₂SO₄. In some embodiments the inorganic salt is a zinc salt such as ZnSO₄, ZnCl₂ or Zn(SO₄)₂. In some embodiments the inorganic salt is a mixture of at least two of a sodium salt, a potassium salt, a calcium salt, an aluminium salt, a lithium salt, a magnesium salt, an iron salt, a copper salt, a nickel salt and a zinc salt.

In some embodiments the height and/or the volume of the glycocalyx of erythrocytes is analysed by atomic force microscopy.

In some embodiments the zeta potential of the erythrocytes is determined using a zeta potential analyser. In some embodiments the zeta potential is analysed by comparing the aggregation tendency of erythrocytes in two different solutions, which both have an about physiological osmolality, but differ in the concentration of the inorganic salt. In such an embodiment a method according to the second aspect includes suspending erythrocytes of the subject in a first solution of an inorganic salt. The first solution is of about physiological osmolality. The method of such an embodiment also includes suspending erythrocytes of the subject in a second solution of an inorganic salt. The second solution is of about physiological osmolality. In some embodiments the first and the second solution are both solutions that contain sodium chloride, which are at least essentially void of potassium chloride—herein also referred to as a sodium chloride solution. If both the first and the second solution are a sodium chloride solution, the first sodium chloride solution has a sodium chloride concentration that is lower than the sodium chloride concentration of the second solution. In one embodiment the sodium chloride concentration in the first sodium chloride solution is about 25 mM or more lower than the sodium chloride concentration of the second sodium chloride solution. In some embodiments comparing the aggregation tendency of erythrocytes in two different solutions of an inorganic salt includes allowing the erythrocytes suspended in the two solutions of an inorganic salt to settle over a period of time. In one embodiment where the inorganic salt is sodium chloride the method includes allowing the erythrocytes suspended in the first sodium chloride solution and the erythrocytes suspended in the second sodium chloride solution to settle over a period of time. The suspended erythrocytes are allowed to settle for a period of time that is sufficient to allow the formation of a supernatant. In embodiments where two different solutions of an inorganic salt are employed for analysing the aggregation tendency of erythrocytes the method further includes detecting the difference in height of the supernatant between the two solutions of an inorganic salt. In embodiments where the inorganic salt is sodium chloride the method generally includes detecting the difference in height of the supernatant between the first and the second sodium chloride solution. Generally the difference in height of the supernatant can also be detected by detecting the difference in height of the settled erythrocytes, i.e. the height of the suspension that still contains erythrocytes, between the two solutions of the inorganic salt, such as the first and in the second sodium chloride solution. An increased difference in height of the supernatant between the two solutions of the inorganic salt, e.g. between the first and the second sodium chloride solution, relative to a threshold value, indicates that the blood pressure of the subject is sensitive to sodium intake.

In some embodiments the method according to the second aspect includes comparing the height or volume of the glycocalyx of the erythrocytes to a threshold value, which may be a control measurement or a predetermined reference value. In embodiments where the inorganic salt is sodium chloride and two suspensions of erythrocytes have been prepared, the method may include comparing the difference in height of the supernatant between the first and the second sodium chloride solution to a control measurement or to a predetermined reference value. In some embodiments the threshold value is based on the difference in height of the supernatant between a corresponding first and a corresponding second sodium chloride solution of erythrocytes of a control sample.

If a reduced height or volume of the glycocalyx of the erythrocytes, relative to a threshold value, and/or a decreased zeta potential of the erythrocytes is detected, a method according to the second aspect may include stratifying the subject for monitoring blood pressure. In embodiments where the inorganic salt is sodium chloride and an elevated difference in height of the supernatant between the first and the second sodium chloride solution has been detected, a method according to the second aspect may include stratifying the subject for monitoring blood pressure.

In some embodiments of the method according to the second aspect the aggregation tendency of erythrocytes is compared in two pairs of two different solutions, with all solutions having an about physiological osmolality. The first pair of solutions contains a first and a second sodium chloride solution. The first and the second solution of this first pair of solutions contain sodium chloride, but differ in the concentration of sodium chloride. In one embodiment the sodium chloride concentration in the first solution is about 25 mM or more lower than the sodium chloride concentration of the second sodium chloride solution of the first pair of solutions. The second pair of solutions contains a first and a second sodium solution. The first solution of this second pair of solutions contains sodium chloride. The second solution of this second pair of solutions contains an inorganic salt different from sodium chloride and is at least essentially void of sodium chloride. In one embodiment the inorganic salt included in the second solution of the second pair of solutions is a potassium salt such as potassium chloride. Erythrocytes of the subject are being suspended in the solutions of both the first and the second pair of solutions. In one embodiment the concentration of sodium chloride in the first solution of the first pair of solutions is at least essentially the same as the concentration of the different inorganic salt in the second solution of the second pair of solutions. In one embodiment the concentration of sodium chloride in the first solution of the second pair of solutions is about 25 mM or more lower than the concentration of the different inorganic salt in the second solution of the second pair of solutions.

In some embodiments of the method according to the second aspect repeated sessions of the method according to the second aspect are carried out. Sessions of the method may be carried out at certain, e.g. predetermined, time intervals.

In a third aspect the present invention provides an in vitro method of analysing whether the blood pressure of a subject is sensitive to sodium intake. The method includes suspending erythrocytes of the subject in a first sodium chloride solution of about physiological osmolality. The method also includes suspending erythrocytes of the subject in a second sodium chloride solution of about physiological osmolality. The first sodium chloride solution has a sodium chloride concentration, which has a value that is about 25 mM or more below the value of the sodium chloride concentration of the second sodium chloride solution. The method further includes allowing the erythrocytes suspended in the first sodium chloride solution and the erythrocytes suspended in the second sodium chloride solution to settle over a period of time. The suspended erythrocytes are allowed to settle for a period of time that is sufficient to allow the formation of a supernatant. The method also includes detecting the difference in height of the supernatant between the first and the second sodium chloride solution. Generally the difference in height of the supernatant can also be detected by detection the difference in height of the settled erythrocytes, i.e. the height of the suspension that still contains erythrocytes, between the first and in the second sodium chloride solution. An increased difference in height of the supernatant between the first and the second sodium chloride solution, relative to a threshold value, indicates that the blood pressure of the subject is sensitive to sodium intake.

In some embodiments the method according to the third aspect includes comparing the difference in height of the supernatant between the first and the second sodium chloride solution to a control measurement or to a predetermined reference value.

In some embodiments the threshold value is based on the difference in height of the supernatant between a corresponding first and a corresponding second sodium chloride solution of erythrocytes of a control sample.

If an increased difference in height of the supernatant between the first and the second sodium chloride solution has been detected, a method according to the third aspect may include stratifying the subject for monitoring blood pressure.

In some embodiments of the method according to the third aspect repeated sessions of the method according to the third aspect are carried out. Sessions of the method may be carried out at certain, e.g. predetermined, time intervals.

In some embodiments the first sodium chloride solution has a sodium chloride concentration that has a value of about 60 mM or more below the value of the sodium chloride concentration of the second sodium chloride solution. In some embodiments the first sodium chloride solution has a sodium chloride concentration of about 100 mM and the second sodium chloride solution has a sodium chloride concentration of about 125 mM. In some embodiments the first sodium chloride solution has a sodium chloride concentration of about 80 mM and the second sodium chloride solution has a sodium chloride concentration of about 140 mM.

In some embodiments of the method according to the third aspect the first and the second sodium chloride solution include a polysaccharide, for example in an amount of about 3% (w/w). If the polysaccharide is dextran, it typically has an average molecular weight of about 70,000 Dalton. Where another polysaccharide is included in the first and the second sodium chloride solution, it may also have an average molecular weight of about 70,000 Dalton. In some embodiments the polysaccharide has a molecular weight of about 70,000 Dalton.

In some embodiments one or both of the first and the second sodium chloride solution include a monosaccharide or a disaccharide, such as sucrose.

In some embodiments the first sodium chloride solution has a sodium chloride concentration of about 125 mM, includes about 3% (w/w) dextran of a molecular weight of about 70,000 Dalton and 50 mM sucrose. In some of these embodiments the second sodium chloride solution has a sodium chloride concentration of about 150 mM and includes about 3% (w/w) dextran of a molecular weight of about 70,000 Dalton. In one embodiment the first sodium chloride solution is an aqueous solution that at least essentially consists of (i) sodium chloride in a concentration of about 110 mM, (ii) dextran of a molecular weight of about 70,000 Dalton in an amount of about 3% (w/w), and (iii) sucrose in a concentration of about 80 mM. In this embodiment the second sodium chloride solution is an aqueous solution that at least essentially consists of (i) sodium chloride in a concentration of about 130 mM (ii) dextran of a molecular weight of about 70,000 Dalton in an amount of about 3% (w/w), and (iii) sucrose in a concentration of about 40 mM. In one embodiment the first sodium chloride solution is an aqueous solution that at least essentially consists of (i) sodium chloride in a concentration of about 125 mM, (ii) dextran of a molecular weight of about 70,000 Dalton in an amount of about 3% (w/w) and (iii) sucrose in a concentration of about 50 mM. In this embodiment the second sodium chloride solution is an aqueous solution that at least essentially consists of (i) sodium chloride in a concentration of about 150 mM and (ii) dextran of a molecular weight of about 70,000 Dalton in an amount of about 3% (w/w).

In a fourth aspect the present invention provides an in vitro method of assessing the risk of occurrence of hypertension in a subject. The method includes suspending erythrocytes of the subject in a first sodium chloride solution of about physiological osmolality. The method also includes suspending erythrocytes of the subject in a second sodium chloride solution of about physiological osmolality. The first sodium chloride solution has a sodium chloride concentration, which has a value that is about 25 mM or more below the value of the sodium chloride concentration of the second sodium chloride solution. The method further includes allowing the erythrocytes suspended in the first sodium chloride solution and the erythrocytes suspended in the second sodium chloride solution to settle over a period of time. The suspended erythrocytes are allowed to settle for a period of time that is sufficient to allow the formation of a supernatant. The method also includes detecting the difference in height of the supernatant between the first and in the second sodium chloride solution. Generally the difference in height of the supernatant can also be detected by detection the difference in height of the settled erythrocytes, i.e. the height of the suspension that still contains erythrocytes, between the first and in the second sodium chloride solution. An increased height of the supernatant relative to a threshold value, indicates an increased risk of occurrence of hypertension.

In some embodiments the method according to the fourth aspect is a method of diagnosing and/or stratifying the risk of developing hypertension.

In some embodiments the method according to the fourth aspect includes comparing the difference in height of the supernatant between the first and the second sodium chloride solution to a control measurement or to a predetermined reference value.

In some embodiments the threshold value is based on the difference in height of the supernatant between a corresponding first and a corresponding second sodium chloride solution of erythrocytes of a control sample.

If an increased difference in height of the supernatant between the first and the second sodium chloride solution has been detected, a method according to the fourth aspect may include stratifying the subject for monitoring blood pressure.

In some embodiments the method of the fourth aspect further includes repeatedly detecting the difference in height of the settled erythrocytes.

In some embodiments of the method according to the fourth aspect the first and the second sodium chloride solution include a polysaccharide, for example in an amount of about 3% (w/w). If the polysaccharide is dextran, it typically has an average molecular weight of about 70,000 Dalton. Where a polysaccharide different from dextran is included in the first and the second sodium chloride solution, it may also have an average molecular weight of about 70,000 Dalton. In some embodiments the polysaccharide has a molecular weight of about 70,000 Dalton.

In some embodiments one or both of the first and the second sodium chloride solution include a monosaccharide or a disaccharide, such as sucrose.

In some embodiments of the method according to the fourth aspect the first sodium chloride solution has a sodium chloride concentration of about 125 mM, includes about 3% (w/w) dextran of a molecular weight of about 70,000 Dalton and 50 mM sucrose. In some of these embodiments the second sodium chloride solution has a sodium chloride concentration of about 150 mM and includes about 3% (w/w) dextran of a molecular weight of about 70,000 Dalton. In one embodiment the first sodium chloride solution is an aqueous solution that at least essentially consists of (i) sodium chloride in a concentration of about 125 mM, (ii) dextran of a molecular weight of about 70,000 Dalton in an amount of about 3% (w/w) and (iii) sucrose in a concentration of about 50 mM. In this embodiment the second sodium chloride solution is an aqueous solution that at least essentially consists of (i) sodium chloride in a concentration of about 150 mM and (ii) dextran of a molecular weight of about 70,000 Dalton in an amount of about 3% (w/w).

In a fifth aspect the invention provides a method of screening one or more individuals for risk or future occurrence of a condition associated with hypertension. The method includes suspending erythrocytes of each of the one or more individuals in a first sodium chloride solution of about physiological osmolality. The method also includes suspending erythrocytes of each of the one or more individuals in a second sodium chloride solution of about physiological osmolality. The first sodium chloride solution has a sodium chloride concentration, which has a value that is about 25 mM or more below the value of the sodium chloride concentration of the second sodium chloride solution. The method further includes allowing the erythrocytes suspended in the first sodium chloride solution and the erythrocytes suspended in the second sodium chloride solution to settle over a period of time. The suspended erythrocytes are allowed to settle for a period of time that is sufficient to allow the formation of a supernatant. The method also includes detecting the difference in height of the supernatant between the first and in the second sodium chloride solution. As indicated above, the difference in height of the supernatant can generally also be assessed by detecting the difference in height of the settled erythrocytes, i.e. the height of the suspension that still contains erythrocytes, between the first and in the second sodium chloride solution. An increased difference in height of the supernatant between the first and the second sodium chloride solution, relative to a threshold value, indicates an increased risk of future occurrence of a condition associated with hypertension.

In some embodiments the method according to the fifth aspect includes comparing the difference in height of the supernatant between the first and the second sodium chloride solution to a control measurement or to a predetermined reference value. In some embodiments the threshold value is based on the difference in height of the supernatant between a corresponding first and a corresponding second sodium chloride solution of erythrocytes of a control sample.

If an increased difference in height of the supernatant between the first and the second sodium chloride solution has been detected, a method according to the fifth aspect may include stratifying the subject for monitoring blood pressure.

In some embodiments the method of the fifth aspect further includes repeatedly detecting the difference in height of the settled erythrocytes.

In some embodiments of the method according to the fifth aspect the first and the second sodium chloride solution include a polysaccharide, for example in an amount of about 3% (w/w).

If the polysaccharide is dextran, it typically has an average molecular weight of about 70,000 Dalton. Where a polysaccharide different from dextran is included in the first and the second sodium chloride solution, it may also have an average molecular weight of about 70,000 Dalton. In some embodiments the polysaccharide has a molecular weight of about 70,000 Dalton.

In some embodiments one or both of the first and the second sodium chloride solution include a monosaccharide or a disaccharide, such as sucrose.

In some embodiments of the method according to the fifth aspect the first sodium chloride solution has a sodium chloride concentration of about 125 mM, includes about 3% (w/w) dextran of a molecular weight of about 70,000 Dalton and 50 mM sucrose. In some of these embodiments the second sodium chloride solution has a sodium chloride concentration of about 150 mM and includes about 3% (w/w) dextran of a molecular weight of about 70,000 Dalton. In one embodiment the first sodium chloride solution is an aqueous solution that at least essentially consists of (i) sodium chloride in a concentration of about 125 mM, (ii) dextran of a molecular weight of about 70,000 Dalton in an amount of about 3% (w/w) and (iii) sucrose in a concentration of about 50 mM. In this embodiment the second sodium chloride solution is an aqueous solution that at least essentially consists of (i) sodium chloride in a concentration of about 150 mM and (ii) dextran of a molecular weight of about 70,000 Dalton in an amount of about 3% (w/w).

In a sixth aspect there is provided a method of monitoring the risk of occurrence of hypertension in a subject. The method includes monitoring the difference in height of the supernatant between a first and a second suspension of erythrocytes of the subject in a sodium chloride solution of about physiological osmolality after having allowed the suspended erythrocytes to settle for a period of time. The first sodium chloride solution has a sodium chloride concentration that is at least about 25 mM lower than the sodium chloride concentration of the second sodium chloride solution. An increased difference in height of the supernatant between the first and the second sodium chloride solution, relative to a threshold value, indicates an increased risk of hypertension.

According to a particular embodiment of the method according to the sixth aspect, the difference in height of the supernatant is monitored at certain, e.g. predetermined, time intervals.

In a seventh aspect the present invention provides a method of assessing the risk of occurrence of a hypertension associated condition in a subject. The method includes detecting at least one of (i) the thickness (height) or the volume of the glycocalyx of erythrocytes of the subject, and (ii) the zeta potential of the erythrocytes, i.e. the degree of electrostatic repulsion between erythrocytes of the subject. If a height or volume of the glycocalyx of the erythrocytes is detected that is lower than a threshold value, the method includes monitoring the subject's blood pressure. If a height or volume of the glycocalyx of the erythrocytes is detected that is about at or above a threshold value the subject's blood pressure generally need not be monitored. If a zeta potential of the erythrocytes is detected that is lower than a threshold value, the method includes monitoring the subject's blood pressure. If a zeta potential of the erythrocytes is detected that is about at or above a threshold value the subject's blood pressure generally need not be monitored. In the method an increased risk of occurrence of a hypertension associated condition is diagnosed if an increased blood pressure relative to a threshold value is detected.

In typical embodiments the method according to the seventh aspect also includes comparing the blood pressure to a control measurement or to a predetermined reference value. An increase in systolic and/or diastolic blood pressure compared to the control measurement indicates that the subject is at an increased risk of occurrence of a hypertension associated condition.

The hypertension associated condition is a condition that may occur as a result of hypertension in the organism of a subject. In some embodiments the hypertension associated condition is arteriosclerosis, cardiac and/or kidney insufficiency, brain bleeding (stroke) or myocardial infarction.

In some embodiments of the method according to the seventh aspect the inorganic salt is a sodium salt such as sodium chloride. In some embodiments a respective sodium salt is Na₂SO₄. In some embodiments a respective sodium salt is Na₂NO₃. In some embodiments the sodium salt is Na₂CO₃. In some embodiments the sodium salt is Na₂HPO₄ NaH₂PO₄ or Na₃PO₄. In some embodiments the inorganic salt is a potassium salt. Examples of a suitable potassium salt include, but are not limited to, KCl, Ka₂NO₃ and K₂SO₄. In some embodiments the inorganic salt is a calcium salt such as CaCl₃, Ca₃(PO₃)₂, Ca(NO₃)₂, CaCO₃ or CaCl₂. In some embodiments the inorganic salt is an aluminium salt, such as AlCl₃ or Al₂(SO₄)₃. In some embodiments the inorganic salt is a magnesium salt such as MgCl₂ or MgSO₄. In some embodiments the inorganic salt is a lithium salt. A suitable lithium salt may for example be LiCl or Li₂SO₄. In some embodiments the inorganic salt is a zinc salt such as ZnSO₄, ZnCl₂ or Zn(SO₄)₂. In some embodiments the inorganic salt is a mixture of at least two of a sodium salt, a potassium salt, a calcium salt, an aluminium salt, a lithium salt, a magnesium salt, an iron salt, a copper salt, a nickel salt and a zinc salt.

In some embodiments of the method according to the seventh aspect the height and/or the volume of the glycocalyx of erythrocytes is analysed by atomic force microscopy.

In some embodiments of the method according to the seventh aspect the zeta potential of the erythrocytes is determined using a zeta potential analyser. In some embodiments the zeta potential is analysed by comparing the aggregation tendency of erythrocytes in two different solutions, which both have an about physiological osmolality, but differ in the concentration of the inorganic salt. In such an embodiment a method according to the first aspect includes suspending erythrocytes of the subject in a first solution of an inorganic salt. The first solution is of about physiological osmolality. The method of such an embodiment also includes suspending erythrocytes of the subject in a second solution of an inorganic salt. The second solution is of about physiological osmolality. In some embodiments the first and the second solution are both solutions of sodium chloride, with the first sodium chloride solution having a sodium chloride concentration that is lower than the sodium chloride concentration of the second sodium chloride solution. In one embodiment the sodium chloride concentration in the first sodium chloride solution is about 25 mM or more lower than the sodium chloride concentration of the second sodium chloride solution. In some embodiments comparing the aggregation tendency of erythrocytes in two different solutions of an inorganic salt includes allowing the erythrocytes suspended in the two solutions of an inorganic salt to settle over a period of time. In one embodiment where the inorganic salt is sodium chloride the method includes allowing the erythrocytes suspended in the first sodium chloride solution and the erythrocytes suspended in the second sodium chloride solution to settle over a period of time. The suspended erythrocytes are allowed to settle for a period of time that is sufficient to allow the formation of a supernatant. In embodiments where two different solutions of an inorganic salt are employed for analysing the aggregation tendency of erythrocytes the method further includes detecting the difference in height of the supernatant between the two solutions of an inorganic salt. In embodiments where the inorganic salt is sodium chloride the method generally includes detecting the difference in height of the supernatant between the first and the second sodium chloride solution. Generally the difference in height of the supernatant can also be detected by detection the difference in height of the settled erythrocytes, i.e. the height of the suspension that still contains erythrocytes, between the two solutions of the inorganic salt, such as the first and in the second sodium chloride solution. An increased difference in height of the supernatant between the two solutions of the inorganic salt, e.g. between the first and the second sodium chloride solution, relative to a threshold value, indicates that the blood pressure of the subject is sensitive to sodium intake.

In an eighth aspect the present invention provides a method of assessing the risk of occurrence of a hypertension associated condition in a subject. The method includes detecting the difference in height of the supernatant between a first and a second suspension of erythrocytes of the subject in a sodium chloride solution of about physiological osmolality after having allowed the suspended erythrocytes to settle for a period of time. The period of time is sufficient to allow the formation of a respective supernatant. The sodium chloride solution of the first suspension of erythrocytes has a sodium chloride concentration that is at least about 25 mM lower than the sodium chloride solution of the second suspension of erythrocytes. Furthermore, if a difference in height of the supernatants between the first and in the second suspension is detected that is increased relative to a threshold value, the method includes monitoring the subject's blood pressure. In the method an increased risk of occurrence of a hypertension associated condition is diagnosed if an increased blood pressure relative to a threshold value is detected.

In typical embodiments the method according to the eighth aspect also includes comparing the blood pressure to a control measurement or to a predetermined reference value.

An increase in systolic and/or diastolic blood pressure compared to the control measurement indicates that the subject is at an increased risk of occurrence of a hypertension associated condition.

The hypertension associated condition is a condition that may occur as a result of hypertension in the organism of a subject. In some embodiments the hypertension associated condition is arteriosclerosis, cardiac and/or kidney insufficiency, brain bleeding (stroke) or myocardial infarction.

In a ninth aspect the present invention provides an in vitro method of analysing whether the blood pressure of a subject is sensitive to sodium intake. The method includes suspending erythrocytes of the subject in a sodium chloride solution of about physiological osmolality. The method also includes suspending erythrocytes of the subject in a solution that contains potassium chloride, which is at least essentially void of sodium chloride—herein also referred to as a potassium chloride solution. The potassium chloride solution is of about physiological osmolality. The concentrations of sodium chloride and potassium chloride in the sodium chloride solution and the potassium chloride solution, respectively, are of at least essentially the same value. The method further includes allowing the erythrocytes suspended in the sodium chloride solution and the erythrocytes suspended in the potassium chloride solution to settle over a period of time. The suspended erythrocytes are allowed to settle for a period of time that is sufficient to allow the formation of a supernatant. The method also includes detecting the difference in height of the supernatant between the sodium chloride solution and the potassium chloride solution. Generally the difference in height of the supernatant can also be detected by detection the difference in height of the settled erythrocytes, i.e. the height of the suspension that still contains erythrocytes, between the first and in the second sodium chloride solution. An increased difference in height of the supernatant between the sodium chloride solution and the potassium chloride solution, relative to a threshold value, indicates that the blood pressure of the subject is sensitive to sodium intake.

In some embodiments the method according to the ninth aspect includes comparing the difference in height of the supernatant between the sodium chloride solution and the potassium chloride solution to a control measurement or to a predetermined reference value.

In some embodiments the threshold value is based on the difference in height of the supernatant between a corresponding sodium chloride solution and a corresponding potassium chloride solution of erythrocytes of a control sample.

If an increased difference in height of the supernatant between the sodium chloride solution and the potassium chloride solution has been detected, a method according to the ninth aspect may include stratifying the subject for monitoring blood pressure.

In some embodiments of the method according to the ninth aspect repeated sessions of the method according to the ninth aspect are carried out. Sessions of the method may be carried out at certain, e.g. predetermined, time intervals.

In some embodiments of the method according to the ninth aspect the sodium chloride solution and the potassium chloride solution include a polysaccharide, for example in an amount of about 3% (w/w). If the polysaccharide is dextran, it typically has an average molecular weight of about 70,000 Dalton. Where another polysaccharide is included in the first and the second sodium chloride solution, it may also have an average molecular weight of about 70,000 Dalton. In some embodiments the polysaccharide has a molecular weight of about 70,000 Dalton.

In some embodiments one or both of the sodium chloride solution and the potassium chloride solution include a monosaccharide or a disaccharide, such as sucrose.

In some embodiments the sodium chloride solution has a sodium chloride concentration of about 125 mM, includes about 3% (w/w) dextran of a molecular weight of about 70,000 Dalton and 50 mM sucrose. In some of these embodiments the potassium chloride solution has a potassium chloride concentration of about 150 mM and includes about 3% (w/w) dextran of a molecular weight of about 70,000 Dalton. In one embodiment the sodium chloride solution is an aqueous solution that at least essentially consists of (i) sodium chloride in a concentration of about 125 mM, (ii) dextran of a molecular weight of about 70,000 Dalton in an amount of about 3% (w/w) and (iii) sucrose in a concentration of about 50 mM. In this embodiment the potassium chloride solution may be an aqueous solution that at least essentially consists of (i) potassium chloride in a concentration of about 125 mM, (ii) dextran of a molecular weight of about 70,000 Dalton in an amount of about 3% (w/w) and (iii) 50 mM sucrose.

In some embodiments of the method according to the ninth aspect the period of time for allowing the suspended erythrocytes to settle is selected in the range from about 45 to about 120 minutes.

In some embodiments the method according to the ninth aspect includes suspending erythrocytes of the subject in a first pair of solutions as described above. The first pair of solutions includes a first solution, being a sodium chloride solution of about physiological osmolality. The first pair of solutions further includes a second solution, being a potassium chloride solution of about physiological osmolality. The method also includes suspending erythrocytes of the subject in a second pair of solutions. The second pair of solutions includes a first solution, being a sodium chloride solution of about physiological osmolality. The second pair of solutions also includes a second solution, being a potassium chloride solution of about physiological osmolality. In one embodiment the concentration of sodium chloride in the first solution of the first pair of solutions is at least essentially the same as the concentration of potassium chloride in the second solution of the second pair of solutions.

In a tenth aspect the present invention provides an in vitro method of assessing the risk of occurrence of hypertension in a subject. The method includes suspending erythrocytes of the subject in a sodium chloride solution of about physiological osmolality. The method also includes suspending erythrocytes of the subject in a potassium chloride solution of about physiological osmolality. The concentrations of sodium chloride and potassium chloride in the sodium chloride solution and the potassium chloride solution are of at least essentially the same value. The method further includes allowing the erythrocytes suspended in the sodium chloride solution and the erythrocytes suspended in the potassium chloride solution to settle over a period of time. The suspended erythrocytes are allowed to settle for a period of time that is sufficient to allow the formation of a supernatant. The method also includes detecting the difference in height of the supernatant between the sodium chloride solution and the potassium chloride solution. Generally the difference in height of the supernatant can also be detected by detection the difference in height of the settled erythrocytes, i.e. the height of the suspension that still contains erythrocytes, between the sodium chloride solution and the potassium chloride solution. An increased height of the supernatant relative to a threshold value, indicates an increased risk of occurrence of hypertension.

In some embodiments the method according to the tenth aspect is a method of diagnosing and/or stratifying the risk of developing hypertension. In some embodiments the method according to the tenth aspect is a method of assessing the risk of occurrence of a hypertension associated condition in a subject. The hypertension associated condition may for instance be at least one of cardiovascular disease, kidney disease, hypertensive retinopathy, dementia, and aortic dissection.

In some embodiments the method according to the tenth aspect includes comparing the difference in height of the supernatant between the sodium chloride solution and the potassium chloride solution to a control measurement or to a predetermined reference value.

In some embodiments the threshold value is based on the difference in height of the supernatant between a corresponding sodium chloride solution and a corresponding potassium chloride solution of erythrocytes of a control sample.

If an increased difference in height of the supernatant between the sodium chloride solution and the potassium chloride solution has been detected, a method according to the tenth aspect may include stratifying the subject for monitoring blood pressure.

In some embodiments the method of the tenth aspect further includes repeatedly detecting the difference in height of the settled erythrocytes.

In some embodiments of the method according to the tenth aspect the sodium chloride solution and the potassium chloride solution include a polysaccharide, for example in an amount of about 3% (w/w). If the polysaccharide is dextran, it typically has an average molecular weight of about 70,000 Dalton. Where a polysaccharide different from dextran is included in the sodium chloride solution and the potassium chloride solution, it may also have an average molecular weight of about 70,000 Dalton. In some embodiments the polysaccharide has a molecular weight of about 70,000 Dalton.

In some embodiments one or both of the sodium chloride solution and the potassium chloride solution include a monosaccharide or a disaccharide, such as sucrose.

In some embodiments of the method according to the tenth aspect the sodium chloride solution has a sodium chloride concentration of about 150 mM and includes about 3% (w/w) dextran of a molecular weight of about 70,000 Dalton. In some of these embodiments the potassium chloride solution has a potassium chloride concentration of about 150 mM and includes about 3% (w/w) dextran of a molecular weight of about 70,000 Dalton. In one embodiment the sodium chloride solution is an aqueous solution that at least essentially consists of (i) sodium chloride in a concentration of about 150 mM and (ii) dextran of a molecular weight of about 70,000 Dalton in an amount of about 3% (w/w). In such an embodiment the potassium chloride solution may be an aqueous solution that at least essentially consists of (i) potassium chloride in a concentration of about 150 mM and (ii) dextran of a molecular weight of about 70,000 Dalton in an amount of about 3% (w/w).

In an eleventh aspect the present invention provides an in vitro method of analysing whether the blood pressure of a subject is sensitive to sodium intake and/or whether the risk of occurrence of hypertension in a subject is increased. The method includes suspending erythrocytes of the subject in in one or more pairs of a first solution and a second solution. Both the first solution and the second solution of each pair of a first solution and a second solution are of about physiological osmolality. Each first solution and each second solution contain at least essentially only one of sodium chloride and potassium chloride. For each pair of first and second solution, the first solution contains sodium chloride. For each pair of first and second solution, the second solution contains sodium chloride or potassium chloride. If a second solution of a pair of solutions contains sodium chloride, it has a sodium chloride concentration that is at least about 25 mM higher than the sodium chloride concentration of the first solution. If a second solution of a pair of solutions contains potassium chloride, it has a concentration of potassium chloride that is of at least essentially the same value as the concentration of sodium chloride in the first solution. The method also includes allowing the suspended erythrocytes in the first and in the second solution of each pair of solutions to settle for a period of time sufficient to allow the formation of a supernatant. Furthermore the method includes detecting the difference in height of the supernatant between the first and the second solution of each pair of solutions. An increased difference in height of the supernatants, relative to a threshold value, indicates that the blood pressure of the subject is sensitive to sodium intake and/or that the is at an increased risk of occurrence of hypertension.

In some embodiments the method according to the eleventh aspect is a method of assessing the risk of occurrence of a hypertension associated condition in a subject. The hypertension associated condition may for instance be one or more of cardiovascular disease, kidney disease, hypertensive retinopathy, dementia, and aortic dissection.

In some embodiments the method according to the eleventh aspect includes comparing the difference in height of the supernatant between the sodium chloride solution and the potassium chloride solution to a control measurement or to a predetermined reference value. In some embodiments the threshold value is based on the difference in height of the supernatant between a corresponding sodium chloride solution and a corresponding potassium chloride solution of erythrocytes of a control sample.

In some embodiments of the method according to the eleventh aspect each of the first and the second solution of a pair of a first solution and a second solution has a concentration of sodium chloride or potassium chloride that is about 100 mM or more.

If an increased difference in height of the supernatant between the sodium chloride solution and the potassium chloride solution has been detected, a method according to the eleventh aspect may include stratifying the subject for monitoring blood pressure.

In some embodiments the method of the eleventh aspect further includes repeatedly detecting the difference in height of the settled erythrocytes.

In some embodiments of the method according to the eleventh aspect the first and the second solution of a pair of a first solution and a second solution further include a polysaccharide, for example in an amount of about 3% (w/w). If the polysaccharide is dextran, it typically has an average molecular weight of about 70,000 Dalton. Where a polysaccharide different from dextran is included in the first and the second solution, it may also have an average molecular weight of about 70,000 Dalton. In some embodiments the polysaccharide has a molecular weight of about 70,000 Dalton.

In some embodiments one or both of the first and the second solution of a pair of a first solution and a second solution include a monosaccharide or a disaccharide, such as sucrose.

In some embodiments of the method according to the eleventh aspect the second solution has a sodium chloride concentration of about 150 mM and includes about 3% (w/w) dextran of a molecular weight of about 70,000 Dalton. In some embodiments of the method according to the eleventh aspect the first solution has a sodium chloride concentration of about 100 mM, includes about 3% (w/w) dextran of a molecular weight of about 70,000 Dalton and 100 mM sucrose. In some of these embodiments the second solution has a potassium chloride concentration of about 100 mM, includes about 3% (w/w) dextran of a molecular weight of about 70,000 Dalton and 100 mM sucrose. In one embodiment the second solution is an aqueous solution that at least essentially consists of (i) sodium chloride in a concentration of about 150 mM and (ii) dextran of a molecular weight of about 70,000 Dalton in an amount of about 3% (w/w). In such an embodiment the second solution may be an aqueous solution that at least essentially consists of (i) potassium chloride in a concentration of about 100 mM, (ii) dextran of a molecular weight of about 70,000 Dalton in an amount of about 3% (w/w) and (iii) 100 mM sucrose.

In some embodiments of the method according to the eleventh aspect the threshold value is based on the difference in height of the supernatant between a corresponding first and a corresponding second solution of erythrocytes of a control sample. In some embodiments of the method according to the eleventh aspect the erythrocytes of the subject have been obtained by sedimentation of cells of blood from the subject, followed by removal of the buffy coat. In some embodiments of the method according to the eleventh aspect suspending erythrocytes in the first sodium chloride solution and in the second sodium chloride solution further includes introducing the erythrocytes into a tube. A respective tube may in some embodiments be a capillary, which has an open end and a sealed end.

In some embodiments of the method according to the eleventh aspect the period of time for allowing the suspended erythrocytes to settle is selected in the range from about 45 to about 120 minutes.

In some embodiments the method according to the eleventh aspect includes suspending erythrocytes of the subject in a first and a second pair of solutions. Each pair of solutions encompasses a first solution and a second solution. For the first pair of solutions, both the first solution and the second solution contain sodium chloride. For the second pair of solutions the first solution contains sodium chloride, and the second solution contains potassium chloride. The concentration of sodium chloride in the first solution of the first pair of solutions is at least essentially the same as the concentration of potassium chloride in the second solution of the second pair of solutions.

In a twelfth aspect the present invention provides an in vitro method of analysing whether the blood pressure of a subject is sensitive to sodium intake and/or whether the risk of occurrence of hypertension in a subject is increased. The method includes suspending erythrocytes of the subject in a first solution and a second solution. Both the first solution and the second solution are of about physiological osmolality. The first solution and the second solution contain at least essentially only one of sodium chloride and potassium chloride. The first solution contains sodium chloride and the second solution contains potassium chloride. The second solution has a potassium chloride concentration that is at least about 25 mM higher than the sodium chloride concentration of the first solution. The method also includes allowing the suspended erythrocytes in the first and in the second solution to settle for a period of time sufficient to allow the formation of a supernatant. Furthermore the method includes detecting the difference in height of the supernatant between the first and the second solution. An increased difference in height of the supernatants, relative to a threshold value, indicates that the blood pressure of the subject is sensitive to sodium intake and/or that the is at an increased risk of occurrence of hypertension.

In some embodiments the method according to the twelfth aspect is a method of assessing the risk of occurrence of a hypertension associated condition in a subject. The hypertension associated condition may for instance be one or more of cardiovascular disease, kidney disease, hypertensive retinopathy, dementia, and aortic dissection.

In some embodiments the method according to the twelfth aspect includes comparing the difference in height of the supernatant between the sodium chloride solution and the potassium chloride solution to a control measurement or to a predetermined reference value. In some embodiments the threshold value is based on the difference in height of the supernatant between a corresponding sodium chloride solution and a corresponding potassium chloride solution of erythrocytes of a control sample.

In some embodiments of the method according to the twelfth aspect the first and the second solution has a concentration of sodium chloride or potassium chloride that is about 100 mM or more.

If an increased difference in height of the supernatant between the sodium chloride solution and the potassium chloride solution has been detected, a method according to the twelfth aspect may include stratifying the subject for monitoring blood pressure.

In some embodiments the method of the twelfth aspect further includes repeatedly detecting the difference in height of the settled erythrocytes.

In some embodiments of the method according to the twelfth aspect the first and the second solution further include a polysaccharide, for example in an amount of about 3% (w/w).

If the polysaccharide is dextran, it typically has an average molecular weight of about 70,000 Dalton. Where a polysaccharide different from dextran is included in the first and the second solution, it may also have an average molecular weight of about 70,000 Dalton. In some embodiments the polysaccharide has a molecular weight of about 70,000 Dalton.

In some embodiments one or both the first and the second solution include a monosaccharide or a disaccharide, such as sucrose.

In some embodiments of the method according to the twelfth aspect the second solution has a potassium chloride concentration of about 125 mM, includes about 3% (w/w) dextran of a molecular weight of about 70,000 Dalton and 50 mM sucrose. In some embodiments of the method according to the twelfth aspect the first solution has a sodium chloride concentration of about 100 mM, includes about 3% (w/w) dextran of a molecular weight of about 70,000 Dalton and 100 mM sucrose. In such an embodiment the second solution may be an aqueous solution that at least essentially consists of, or consists of, (i) potassium chloride in a concentration of about 150 mM and (ii) dextran of a molecular weight of about 70,000 Dalton in an amount of about 3% (w/w).

In some embodiments of the method according to the twelfth aspect the threshold value is based on the difference in height of the supernatant between a corresponding first and a corresponding second solution of erythrocytes of a control sample. In some embodiments of the method according to the twelfth aspect the erythrocytes of the subject have been obtained by sedimentation of cells of blood from the subject, followed by removal of the buffy coat. In some embodiments of the method according to the twelfth aspect suspending erythrocytes in the first and in the second solution further includes introducing the erythrocytes into a tube. A respective tube may in some embodiments be a capillary, which has an open end and a sealed end.

In some embodiments of the method according to the twelfth aspect the period of time for allowing the suspended erythrocytes to settle is selected in the range from about 45 to about 120 minutes.

In a thirteenth aspect the invention provides a kit of parts. The kit includes a first and a second container as well as a pair of tubes. The first container includes a sodium chloride solution. The second container includes a sodium chloride solution, which has a sodium chloride concentration that is higher than the sodium chloride concentration of the sodium chloride solution in the first container.

In some embodiments the sodium chloride solution in the second container of the kit has a sodium chloride concentration that is at least about 25 mM higher than the sodium chloride concentration in the first container. In some embodiments the sodium chloride solution in each of the first and the second container is of about physiological osmolality. The sodium chloride solution in each of the first and the second container has in some embodiments a sodium chloride concentration of about 100 mM or more.

In some embodiments the first and the second container further include a monosaccharide such as for instance glucose.

In some embodiments of the kit according to the thirteenth aspect the sodium chloride solutions in the first and in the second container include a polysaccharide, for example in an amount of about 3% (w/w). If the polysaccharide is dextran, it typically has an average molecular weight of about 70,000 Dalton. Where a polysaccharide different from dextran is included in the sodium chloride solutions, it may also have an average molecular weight of about 70,000 Dalton. In some embodiments the polysaccharide has a molecular weight of about 70,000 Dalton.

In some embodiments of the kit one or both of the first and the second sodium chloride solution include a monosaccharide or a disaccharide, such as sucrose.

In some embodiments the sodium chloride solution in the first container of the kit has a sodium chloride concentration of about 125 mM, includes about 3% (w/w) dextran of a molecular weight of about 70,000 Dalton and 50 mM sucrose. In some of these embodiments the the sodium chloride solution in the second container of the kit has a sodium chloride concentration of about 150 mM and includes about 3% (w/w) dextran of a molecular weight of about 70,000 Dalton. In one embodiment the sodium chloride solution in the first container is an aqueous solution that at least essentially consists of (i) sodium chloride in a concentration of about 125 mM, (ii) dextran of a molecular weight of about 70,000 Dalton in an amount of about 3% (w/w) and (iii) sucrose in a concentration of about 50 mM. In this embodiment the sodium chloride solution in the second container is an aqueous solution that at least essentially consists of (i) sodium chloride in a concentration of about 150 mM and (ii) dextran of a molecular weight of about 70,000 Dalton in an amount of about 3% (w/w).

In a fourteenth aspect the invention provides a kit of parts. The kit includes one or more pairs of a first and a second container, as well as a pair of tubes. A first container includes a first solution and a second container includes a second solution. Each first solution and each second solution contain at least essentially only one of sodium chloride and potassium chloride. For each pair of a first and a second container, the first solution includes sodium chloride, and the second solution includes sodium chloride or potassium chloride. A second solution that contains potassium chloride has a concentration of potassium chloride that is of at least essentially the same value as the concentration of sodium chloride in the first solution.

The second container includes a potassium chloride solution, which has a potassium chloride concentration that is of at least essentially the same value as the sodium chloride concentration of the sodium chloride solution in the first container.

In some embodiments the first and the second solution of one pair of a first and a second container are of about physiological osmolality. In some embodiments the first and the second solution of each pair of a first and a second container are of about physiological osmolality. The first and the second solution have in some embodiments a concentration of sodium chloride and of potassium chloride, respectively, of about 100 mM or more.

In some embodiments of the kit according to the fourteenth aspect, for one or more pairs of a first and a second container, a second solution containing sodium chloride has a sodium chloride concentration that is at least about 25 mM higher than the sodium chloride concentration of the first solution.

In some embodiments the first and the second container further include a monosaccharide such as for instance glucose. In some embodiments the first and the second container include a disaccharide such as sucrose.

In some embodiments of the kit according to the fourteenth aspect, in one or more pairs of a first and a second container the first and the second solution further contain a polysaccharide. In some embodiments each of the first and the second solution further contain a polysaccharide, i.e. for each pair of a first and a second container.

In some embodiments the first and the second solution of one or more pairs of a first and a second container include a polysaccharide, for example in an amount of about 3% (w/w). If the polysaccharide is dextran, it typically has an average molecular weight of about 70,000 Dalton. Where a polysaccharide different from dextran is included in the sodium chloride solutions, it may also have an average molecular weight of about 70,000 Dalton. In some embodiments the polysaccharide has a molecular weight of about 70,000 Dalton.

In some embodiments of the kit according to the thirteenth aspect each of the tubes of the pair of tubes is a capillary, the capillary having an open end and a sealable end.

In a fourteenth aspect the invention relates to the in-vitro use of a kit of parts according to the twelfth aspect or according to the thirteenth aspect for assessing the risk of occurrence of hypertension in a subject or for assessing the risk of occurrence of a hypertension associated condition in a subject.

In a fifteenth aspect the invention relates to the in-vitro use of a kit of parts according to the twelfth aspect or according to the thirteenth for assessing the risk of occurrence of hypertension in a subject or for assessing the risk of occurrence of a hypertension associated condition in a subject.

The summary of the invention described above is non-limiting and other features and advantages of the invention will be apparent from the following detailed description of the invention, and from the claims.

### BRIEF DESCRIPTION OF THE DRAWINGS

FIG. 1 illustrates, without being bound by theory, an assumption on the possible basic principle underlying a method as disclosed herein. An erythrocyte is carrying a negatively charged boundary layer, the glycocalyx, which attracts positive charges and repels negative charges.

FIG. 2 illustrates the glycocalyx present on red blood cells.

FIG. 3 depicts the sedimentation of aggregated erythrocytes over a time interval from 60 to 120 minutes. The figure can also serve as an illustration on determining erythrocyte salt sensitivity (ESS).

FIG. 4 depicts the sodium dependency of the negative zeta potential—detected by the height of the supernatant after allowing erythrocytes to settle.

FIG. 5 depicts the detection of results after allowing erythrocytes of samples from two volunteers to settle. Tubes depicted on the right in each case contained 125 mM sodium (L₁), and tubes depicted on the left contained 150 mM sodium (L₂). At the bottom of each figure the individual ESS value is calculated.

FIG. 6 depicts the erythrocyte salt sensitivity of 12 healthy individuals. The grey bar in the center of the figure shows the mean of all volunteers including the standard error. “Group A” consists of 3 volunteers who are statistically significantly less sensitive to salt than the average (p<0.01). “Group B” consists of 3 volunteers who are statistically significantly more sensitive to salt than the average (p<0.01).

FIG. 7 depicts an image of erythrocytes obtained by atomic force microscopy.

FIG. 8 schematically illustrates the visualisation of the glycocalyx of erythrocytes by differential imaging as shown in FIG. 9.

FIG. 9 depicts atomic force microscopy images of (A) erythrocytes before the removal of the glycocalyx and (B) erythrocytes after removal of the glycocalyx, as well as (C) a differential image (A-B) showing only the glycocalyx.

FIG. 10 depicts data (glycocalyx height) of monitoring by atomic force microscopy the removal of the glycocalyx on erythrocytes by means of heparinase.

FIG. 11 illustrates the progress of glycocalyx removal on erythrocytes depicted by atomic force microscopy.

FIG. 12 depicts data (glycocalyx volume) of monitoring, by atomic force microscopy, the removal of the glycocalyx on erythrocytes by means of heparinase. Volume is indicated in femtoliters, i.e. units corresponding to 10⁻¹⁵ liters.

FIG. 13 depicts atomic force microscopy images of erythrocytes after exposure to intact endothelium.

FIG. 14 depicts atomic force microscopy images of erythrocytes after exposure to glycocalyx-depleted endothelium.

FIG. 15 shows the interrelation between intactness of endothelial and erythrocyte glycocalyx. Glycocalyx height of erythrocytes exposed to intact endothelium (black) is substantially greater than glycocalyx height of erythrocytes exposed to glycocalyx-depleted endothelium (white). The value of glycocalyx-depleted erythrocytes exposed to intact endothelium is depicted as a white square.

FIG. 16 illustrates a comparison of the difference in height of supernatant after erythrocytes have been allowed to settle for 60 minutes, which were suspended in a solution of 125 mM sodium chloride. The erythrocytes had been exposed (A) to intact endothelium and (B) to glycocalyx depleted endothelium.

FIG. 17 depicts the rate of sedimentation of erythrocytes which were allowed to settle after exposure to intact endothelium (black bar) and to glycocalyx depleted endothelium (white bar).

FIG. 18 depicts the stiffness and the thickness (height) of the glycocalyx, as determined by atomic force microscopy, of blood vessels after 5 days of treatment with chronic low sodium (black dots), and treatment with chronic high sodium (white dots). As can be taken from the figure, constant exposure to chronic high sodium levels causes the endothelial glycocalyx to shrink and stiffen.

FIG. 19 shows the erythrocyte sodium sensitivity (triple measurements) of 61 study participants. The value is 4.28±0.19 (n=61). Five study participants explicitly indicated to be on a low-salt diet (white bars).

FIG. 20 depicts a comparison of two embodiments of a salt blood test according to the present disclosure. An embodiment using capillary blood (fingertip) was compared to an embodiment using venous puncture (venous blood) in 6 test persons. Paired study, i.e. venous blood and capillary blood were taken from the same person and analysed on the same day. The correlation of a linear regression line (order 1; not shown in graph) is 0.91.

FIG. 21 illustrates a method disclosed herein, in which salt sensitivity is analysed in terms of sodium selectivity. Capillary blood from the fingertip was obtained from 12 study participants. The Na⁺ over K⁺ selectivity i.e. the ratio of erythrocyte sedimentation rates in 125 Na⁺ over 125 K⁺ solutions (ESSK=L125 Na⁺/L125 K⁺) are shown. A mean value of 3.11±0.21 indicates that in average Na⁺ binds about 3-times more selective as compared to K⁺.

FIG. 22 shows the frequency distribution of the erythrocyte sodium sensitivity in a cohort of 61 study participants.

### DETAILED DESCRIPTION OF THE BEST MODE OF THE INVENTION

Provided are methods of analysing the sensitivity of a subject's blood pressure to sodium intake and of predicting the susceptibility of a subject to develop hypertension. Methods are described that can be carried out rapidly and easily. A method or use provided in this specification may in some embodiments be a method for risk assessment, a method for diagnosis or prognosis of the occurrence of hypertension and/or a hypertension related condition in a subject. In some embodiments a method or use described herein relates to the assessment of prehypertension in a subject, i.e. a subject with a systolic blood pressure of values in the range from about 120 to about 139 mm Hg and/or a diastolic blood pressure of values in the range from about 80 to about 89 mm Hg.

A method or use described herein may be used in diagnosis or prognosis assessment of hypertension and/or a hypertension related condition in a subject, generally a mammalian subject. A respective use or method can provide an indication of a risk of developing hypertension in a subject. The respective method or use may also be used to determine whether the blood pressure of a subject is sensitive to sodium intake. A positive assessment in this regard will generally be an indication that the vascular system of the respective subject is sensitive to changes in sodium intake. Put differently, a subject's blood pressure is sensitive to sodium intake, if an intake of sodium, generally by ingestion of food with a high amount of sodium, can lead to an increase of the subject's blood pressure. This term is generally to be understood as a relative term in that a subject's blood pressure is sensitive to sodium intake if an intake of a given amount of sodium, typically per kg body weight, causes an increase of blood pressure that is higher than the increase of blood pressure induced by the same amount of sodium in the organism of an average individual with a comparable age. Further, a method or use as disclosed herein may also be used in stratifying a subject for hypertension prevention measures or for hypertension therapy. An “individual” or “subject” as used herein refers to any mammal, including e.g. a rabbit, a mouse, a Guinea pig, a hamster, a dog, a pig, a cow, a sheep, a horse, a macaque, a gibbon and a human. A respective mammal may in some embodiments be a veterinary animal such as a farm animal, a domestic animal or laboratory animal. Where the subject is a human, the subject may be a patient.

The present invention is based on the surprising finding that the functional state of the glycocalyx of erythrocytes reflects the functional state of the endothelial glycocalyx. Therefore the functional state of both the glycocalyx of erythrocytes and the endothelial glycocalyx are useful prognostic parameters for a disposition of hypertension. The term “hypertension” as used herein refers to high arterial blood pressure. Generally high arterial blood pressure is characterized by a systolic blood pressure that is consistently over 140 mm Hg and/or a diastolic blood pressure that is consistently over 90 mm Hg. If either or both of the systolic blood pressure and the diastolic blood pressure are too high, a subject has hypertension.

A subject to be diagnosed, stratified, screened or for whom/which an assessment is to be made is generally a subject that does not or not yet suffer from a hypertension induced condition. Typically the subject does not have a history of a hypertension induced condition such as a vascular disease, e.g. stroke or myocardial infarction. Typically the subject does not suffer from atherothrombosis. In some embodiments the subject is not suffering from an inflammatory condition of the vascular system. In some embodiments the subject does not (yet) suffer from hypertension. In some embodiments the subject does not suffer from an acute disease or from atherothrombosis.

The erythrocytes used in a method or use according to this disclosure have been obtained from the individual before carrying out the method/use. The erythrocytes may in some have been kept at room temperature, i.e. about 18° C. The erythrocytes may also have been kept at 37° C. The erythrocytes may also have been kept at a temperature in the range between 18° C. and 37° C. The erythrocytes used in a method or use described herein may have been isolated from a blood sample obtained the individual before carrying out the method/use and subsequen have been kept at a temperature in the range from about 18° C. to about 37° C. Before isolating the erythrocytes the blood sample may likewise have been kept at 37° C. The sample may also have been kept at a temperature in the range between 18° C. and 37° C. In some embodiments the erythrocytes isolated from the blood sample may have been kept at a temperature in the range from about 2° C. to about 37° C., such as from about 4° C. to about 37° C. or below. In some embodiments the the erythrocytes may have been kept at about 32° C. or below. In some embodiments the erythrocytes may have been kept at a temperature of about 25° C. or below. As an illustrative example, a whole blood sample may be kept at about 25° C. or below.

The word “about” as used herein refers to a value being within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects according to the invention. In this context “about” may refer to a range above and/or below of up to 10%. The word “about” refers in some embodiments to a range above and below a certain value that is up to 5%, such as up to up to 2%, up to 1%, or up to 0.5% above or below that value. In one embodiment “about” refers to a range up to 0.1% above and below a given value.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

In some embodiments the blood sample has been taken on the same day or on the previous day, such as about 48 hours or about 42 hours, before the method described herein is being carried out. In some embodiments the blood sample has been taken about 36 hours before carrying out a methoddescribed herein. In some embodiments the blood sample has been taken about 30 hours before carrying out a respective method. In some embodiments the blood sample has been taken about 28 hours or about 24 hours before the method is being carried out. In some embodiments the blood sample has been taken about 18 hours before carrying out a method or use as described herein. In some embodiments the blood sample has been taken about 15 hours before the method is being carried out. The blood sample may also have been taken about 12 hours earlier. The blood sample may in some embodiments have been taken about 6 hours or less earlier, i.e. before carrying out a method of this disclosure. In some embodiments the blood sample has been taken about 2 hours or less before carrying out the method or use. In some embodiments the blood sample has been obtained about 30 minutes or less before carrying out the method or use.

A method according to this disclosure can be carried out on enriched or isolated erythrocytes of the subject. The term “subject” as used herein, also addressed as an individual, refers to a human or non-human animal, generally a mammal. A subject may be a mammalian species such as a rabbit, a mouse, a rat, a Guinea pig, a hamster, a dog, a cat, a pig, a cow, a goat, a sheep, a horse, a monkey, an ape or a human. Thus, the methods, uses and compositions described in this document are applicable to both human and veterinary disease. As explained in more detail below, the sample has been obtained from the subject. Further, while a subject is typically a living organism, a method or use described in this document may also be used in post-mortem analysis. Where the subject is a living human who is receiving medical care for a disease or condition, it is also addressed as a “patient”.

By the use of the term “enriched” is meant that the erythrocytes constitute a significantly higher fraction (such as 2-5 fold) of all cells present in a sample of interest such as a cell suspension than in the natural source from which the sample was obtained. The cell may also constitute a significantly higher fraction than in an organism, whether normal or diseased. This can most conveniently be achieved by preferential reduction in the amount of other cells present, although a preferential increase in the amount of erythrocytes or a combination of the two may likewise be applied. However, it should be noted that enriched does not imply that there are no other cells present. The term merely defines that the relative amount of erythrocytes has been significantly increased. The term significant here is used to indicate that the level of increase is useful to the person achieving such an increase, and generally means an increase relative to other amino acid or nucleic acid sequences of about at least 2-fold, for example at least about 5- to 10-fold or even more. The term is meant to cover only those situations in which man has intervened to increase the proportion of erythrocytes.

The term “isolated” indicates that the cell or cells has/have been removed from its/their normal physiological environment, e.g. a natural source. An isolated cell or isolated cells may for instance be included in a different medium such as an aqueous solution than provided originally, or placed in a different physiological environment. Typically isolated cells constitute a higher fraction of the total cells present in their environment, e.g. suspension, than in the environment from which they were taken. The term “isolated” does not imply that erythrocytes are the only cell type present, but that these cells are essentially free, e.g. about 80-90% pure or more, of other cells, respectively, in particular cells naturally associated with these cells.

Erythrocytes of the subject can conveniently be obtained from a blood sample of the subject since the majority of cells in blood are erythrocytes. Thus centrifugation of blood, for instance at 500×g, will for instance result in a cell pellet that consists mainly of erythrocytes, which can be recovered after removal of the supernatant. Most of the white blood cells and platelets are found in a so called buffy coat, a whitish sediment forming a layer above the erythrocyte pellet. This buffy coat can thus likewise be removed. Where desired the obtained cell pellet may be washed once or several times by resuspending in a suitable buffer, centrifugation and removal of supernatant. Any other method of enriching or isolating red blood cells may likewise be employed in order to obtain erythrocytes of a subject. An illustrative further example of obtaining erythrocytes is cell chromatography.

Over the past years clinical detection methods such as the method of pulse wave analysis (Gurovich A N & Braith R W, Hypertens Res (2011) 34, 166-169) or various imaging technologies (e.g. intima media thickness) have been refined to a degree that the structural/functional state of the vessel system can be conceived in detail. Apart from the relative complexity of these examinations, which does not permit a widespread use throughout the population for purely preventive purposes, these modern methods do only detect the state of the blood vessels once already visible (structural) changes have occurred. Therefore, these methods are not suitable for the early diagnosis of a disposition for hypertension. Furthermore such elaborate methods are relatively complex since imaging of a subject is required.

As explained above, a method described herein makes use of the finding that the functional state of the glycocalyx of erythrocytes can be used as a prognostic parameter to identify a subject's disposition to hypertension. It has previously been suggested that electrostatic repulsive force prevents aggregation of erythrocytes and that Neuraminidase treatment increases the degree of aggregation of erythrocytes (Jan, K.-M., and Chien, S., J. Gen Physiol. (1973) 61, 638-654). The present inventor verified this observation and developed diagnostic methods that can be used as a quick test to inter alia identify subjects that are susceptible to develop hypertension as well as susceptible to cardiovascular diseases, and to determine whether an individual's blood pressure is different in sodium sensitivity, such as more sensitive or less sensitive to sodium intake than an average individual of comparable health and age.

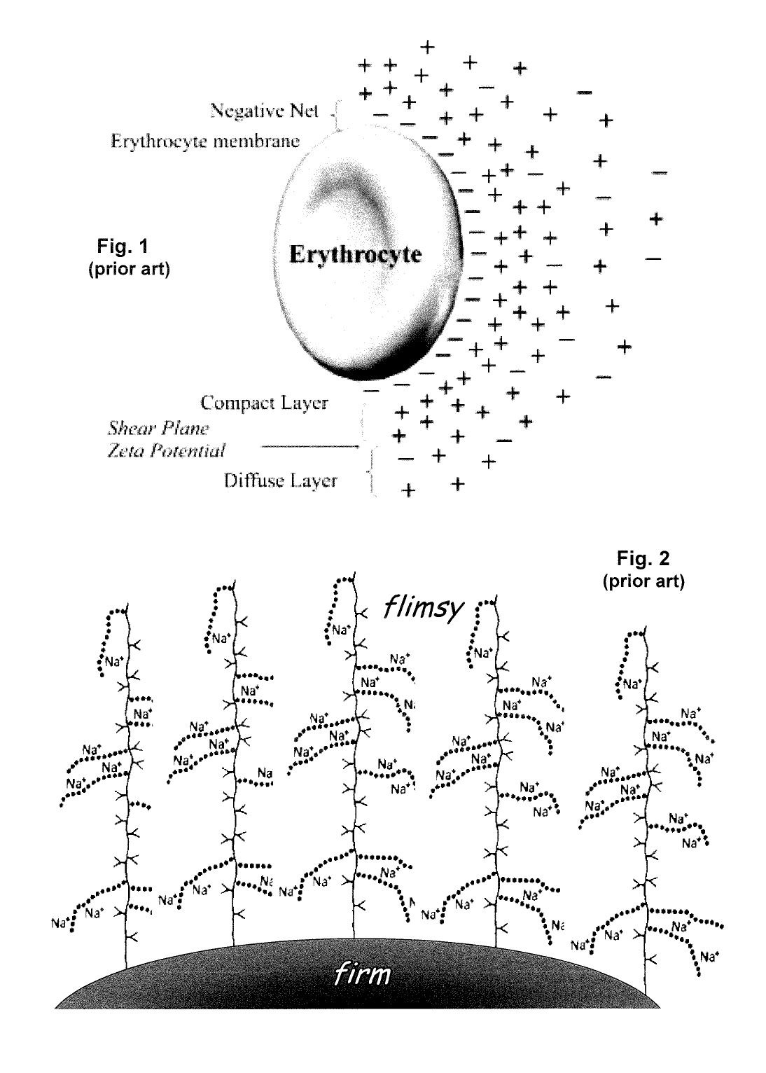

As is well known in the art, the inner wall of blood vessels is lined with a layer of endothelial cells, which are of high importance for the function of blood vessels. On this endothelial surface there is a so-called endothelial glycocalyx (eGC), an anionic glycoprotein layer of about 500 nm in thickness and rich in water. The eGC participates in the regulation of vascular permeability, in the control of flow- and pressure-induced mechanotransduction of the endothelium and may play a crucial role in the pathogenesis of inflammation. Proteoheparan sulphate macromolecules, anchored in the plasma membrane expose negatively charged glycosaminoglycan side chains with binding sites for inorganic cations. This eGC is capable of temporarily buffering sodium of the blood. Recent research results support the view that the functional state of this eGC has a determining influence on the transport of sodium from the blood stream into tissue (Oberleithner, H., et al., Pflügers Arch (2011) 462, 519-528). In other words, an intact eGC defines a functional barrier for the resorption of sodium ions that have entered the blood. The ions can thus be eliminated via the kidneys and do not have to take a route through the entire organism, involving a transient deposit in tissues and organs (cf. FIG. 1). The eGC may play a prominent role as a buffer barrier for sodium.

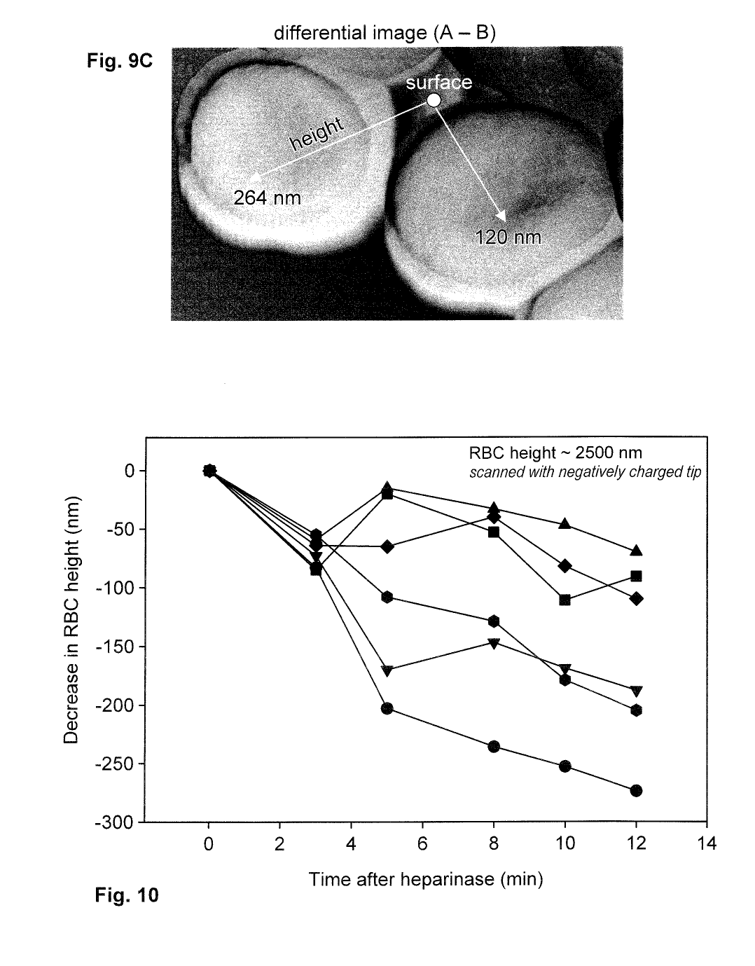

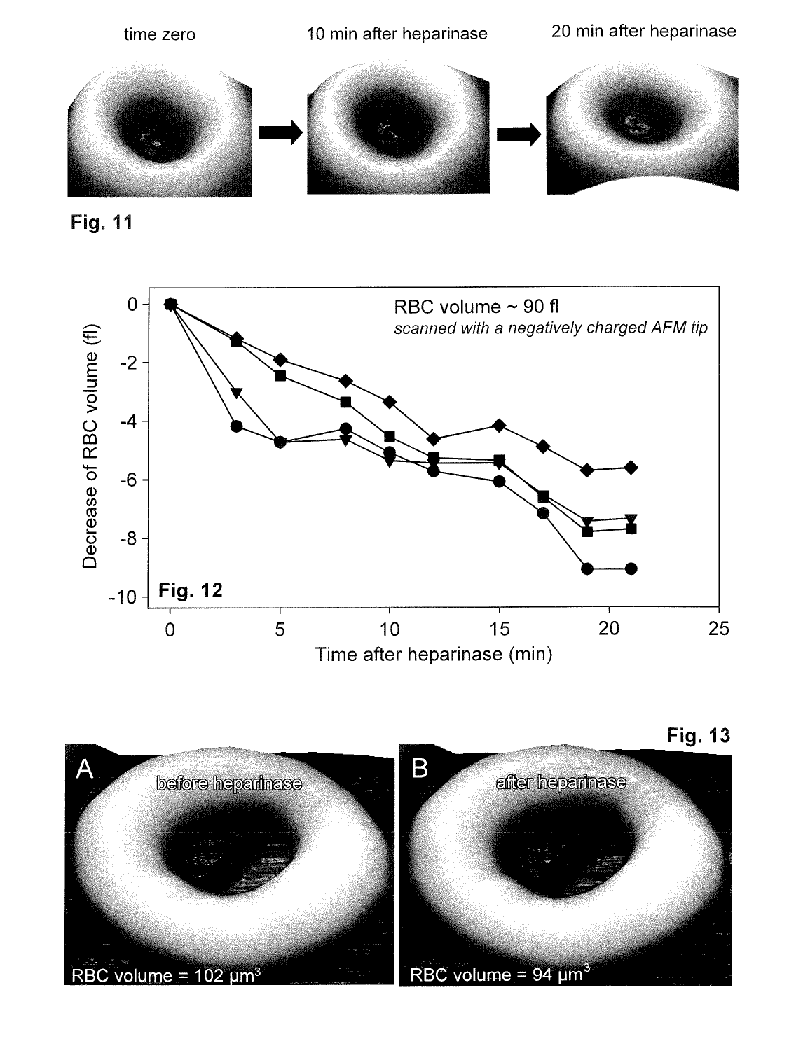

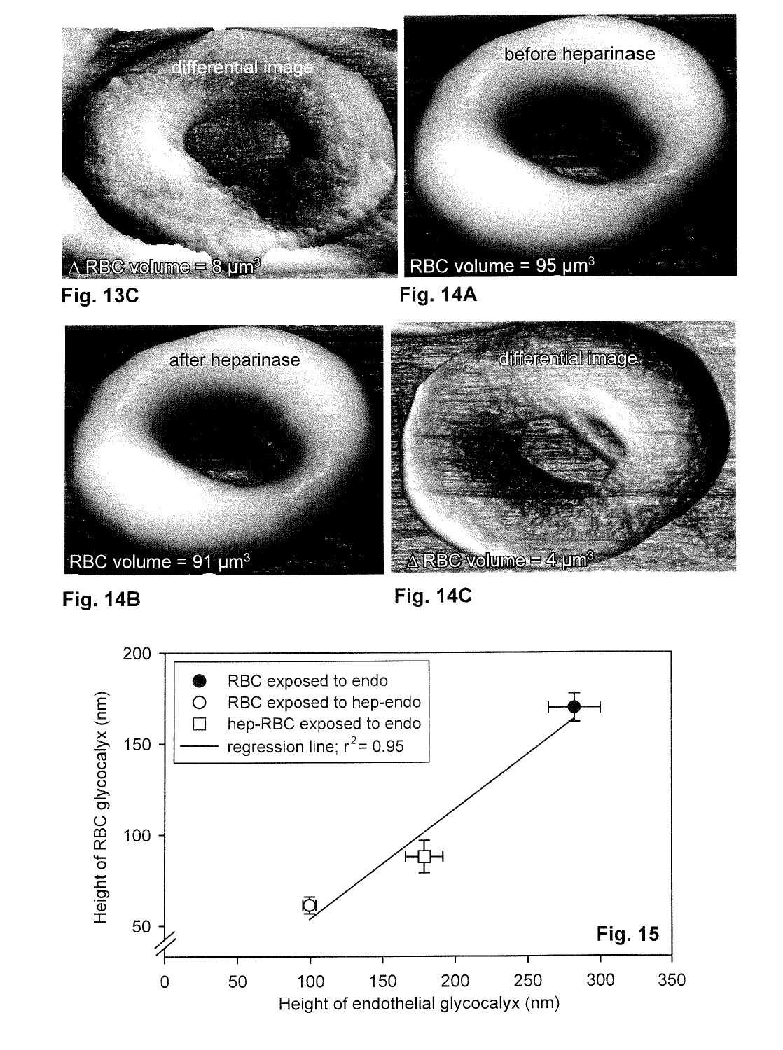

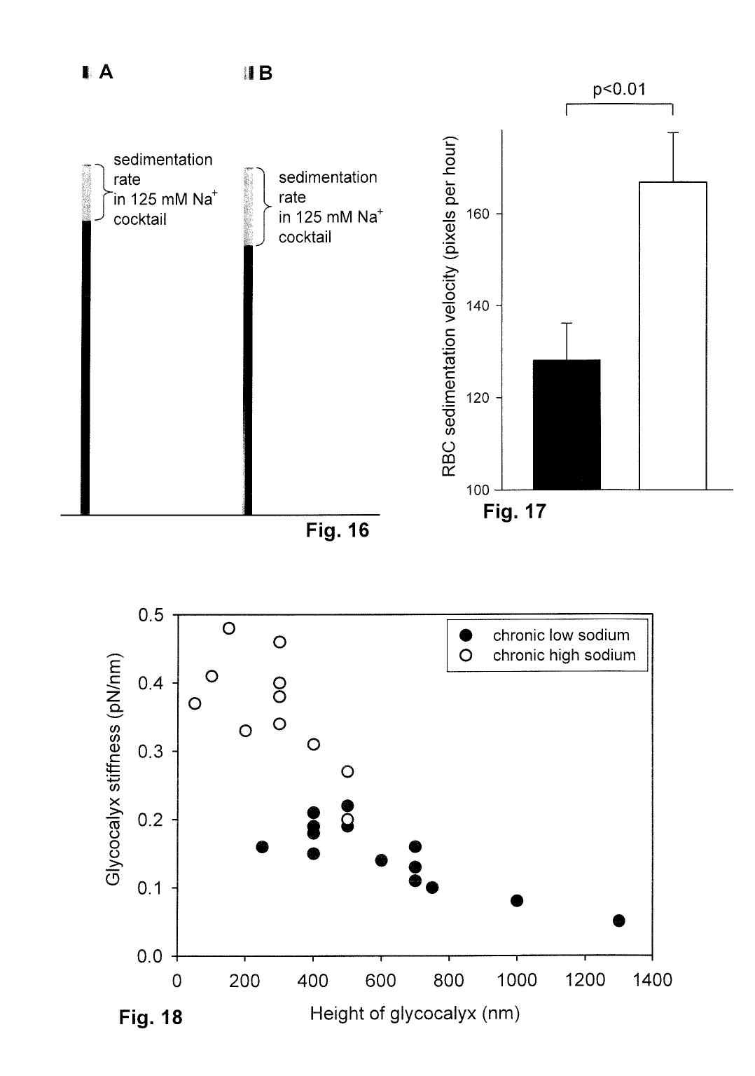

As illustrated in FIG. 1 and FIG. 2, erythrocytes likewise have a glycocalyx, which defines a soft surface of macromolecules surrounding an erythrocyte. As can be taken from FIG. 9C, FIG. 10 and FIG. 15, the glycocalyx thickness of red blood cells is much higher than the few nanometers previously assumed, resulting in a contribution of the glycocalyx to the volume of erythrocytes that cannot be disregarded (FIG. 12). The negative surface charge of residues of the glycocalyx generates an electrostatic repulsive force. Without being bound by any particular theory it can thus be assumed that changes to the glycocalyx thickness will also modulate the charges per membrane surface area. Such changes will affect the net charge of an erythrocyte and thereby the repulsive force it exerts onto other erythrocytes. As erythrocytes form a cell suspension, a zeta potential can be detected, which characterizes the degree of repulsion between cells, and thus the stability of the corresponding cell suspension. The higher the zeta potential the less likely is aggregation to occur in the cells suspension. In a method as described herein an indirect indication of the zeta potential is used, namely the rate of aggregation of erythrocytes. This rate of aggregation can conveniently be assessed by allowing the erythrocytes to settle at different salt concentrations.

In a method disclosed herein erythrocytes are being suspended in two solutions. These two solutions may in some embodiments be two or more sodium chloride solutions. Both sodium chloride solutions are of about physiological osmolarity so that the erythrocytes remain intact while the method is being carried out. In some embodiments these two solutions may include a sodium chloride solution and a potassium chloride solution.

It is understood that in the context of a method or use described herein both osmolarity and osmolality may be detected in order to adjust suitable conditions with regard to solution used to suspend erythrocytes therein. Suitable conditions in this context are conditions that avoid the formation of osmotic pressure into or out of erythrocytes from the subject. Where two salt solutions of different salt concentrations are used, the osmotic conditions to which the erythrocytes are exposed are adjusted to be at least essentially comparable, including at least essentially identical. Osmolarity is expressed as the number of solute particles per unit of volume, e.g. liter, of solvent, whereas osmolality is expressed as the number of solute particles per unit of mass, e.g. kilogram, of solvent, typically water. Osmolality can conveniently be measured using an osmometer. As the skilled person is well aware, osmolarity is thus affected by changes in water content, temperature and pressure, while osmolality is independent of temperature and pressure.

In embodiments where two sodium chloride solutions are being employed, the two sodium chloride solutions differ in their sodium chloride concentration by a value of about 25 mM or more. As an illustrative example the two solutions may differ in their sodium chloride concentration by about 40 mM or more. As a result of the difference in sodium chloride concentration the number of electrolytes present in the two solutions differs and the repulsive forces between erythrocytes in the two solutions likewise differ. Thus a difference in the rate of aggregation exists between erythrocytes in the two solutions. Accordingly, if erythrocytes with approximately the same cell number per millilitre are allowed to settle for a limited period of time in solutions with different sodium chloride concentrations the erythrocytes in the two solutions settle at a different rate. The present inventor has observed a correlation in that the less repulsive forces exist between the erythrocytes the greater is the difference in aggregation tendency. Thus if erythrocytes with approximately the same cell number per millilitre are allowed to settle for a limited period of time in two solutions with different sodium chloride concentrations, the erythrocytes in the solutions with the higher sodium chloride concentration will settle at a higher rate than the erythrocytes in the solutions with the lower sodium chloride concentration. In some embodiments the two sodium chloride solutions differ in their sodium chloride concentration by about 50 mM or more. In some embodiments the two sodium chloride solutions differ in their sodium chloride concentration by about 70 mM or more.

Generally in a method disclosed herein any means to detect erythrocyte sedimentation velocity may be employed. Typically erythrocyte sedimentation velocity is measured in aqueous solution, which may advantageously be selected to be an isosmotic electrolyte solution. Where two aqueous solutions are being used, which differ in their sodium salt concentration by about 25 mM or more, sedimentation velocities in these two solutions can be detected and compared. A ratio of the erythrocyte sedimentation velocity in a solution of a higher sodium salt concentration over erythrocyte sedimentation velocity in a solution of a lower sodium salt concentration can be determined, herein also called the erythrocyte sodium sensitivity (ESS). This erythrocyte sodium sensitivity is inversely related to erythrocyte sodium buffer capacity (cf. above). In one embodiment the ratio of the erythrocyte sedimentation velocities in a solution of a sodium salt such as sodium chloride of 150 mM and 125 mM is being determined. In such an embodiment the ESS is expressed as the ratio of the erythrocyte sedimentation velocities of 150 mM over 125 mM Na⁺ solutions. the Na⁺ solutions.

In some embodiments a sodium chloride solution and a potassium chloride solution are being employed. Without being bound by theory, the following explanations may provide an illustration on how the method developed by the inventor can be envisaged to find a physiological background. Erythrocyte sedimentation rate in a defined electrolyte solution not only depends on ion strength but also on the ion species. There are difference in the properties of the specific ion, such as the size of the ion, the charge density on its surface and binding capacity of water molecules to its surface, which may be envisaged to be involved. A hypothetic model on the effect of an ion's charge density in aqueous solution, its enthalpy of hydration, and the resulting effects on interactions in biological systems has been given by Collins (Biophysical Journal (1997) 72, 65-76).

Na⁺ is a so-called kosmotropic ion, also called antichaotropic, i.e. a small cation with high electrical charge density at its surface. Because of this specific property a Na⁺ ion is thought to bind water molecules tightly (“water maker”). Due to the specific properties and the high concentration of Na⁺ in extracellular fluids including human plasma this cation is the major binding partner for the negatively charged glycocalyx. The electrical surface charge properties of the erythrocyte glycocalyx (zeta potential) determine erythrocyte sedimentation rate in defined protein-free electrolyte solution. As described elsewhere herein the erythrocyte sodium sensitivity (ESS) can be determined by using specific electrolyte solutions of two different Na⁺ concentrations. ESS is an indirect measure of vascular sodium sensitivity. A large ESS indicates a poor Na⁺ buffering power of the glycocalyx and vice versa. From the erythrocyte sodium sensitivity a conclusion can be drawn on the sodium buffering capacity of the erythrocyte glycocalyx, and indirectly on that of the endothelial glycocalyx.

An extention of this hypothesis is the comparison of Na⁺ and K⁺ binding to the negatively charged glycocalyx. In contrast to kosmotropic sodium, K⁺ is classified as being chaotropic (Collins, K. D., 1997, supra). K⁺ is thought to be larger in size compared to Na⁺, with a surface charge density that is smaller, and water binding being weak. These theoretic conclusions may explain, why it is unexpectedly possible to assess a subject's blood pressure sensitivity by comparing erythrocyte sedimentation rates in two or more separate electrolyte solutions, each of which essentially containing only one of Na⁺ and K⁺. One solution used contains Na⁺, and one solution K⁺. The solutions containing Na⁺ and K⁺, respectively, have at least essentially the same ionic strength of the respective ion. In one embodiment the solutions containing Na⁺ and K⁺, respectively, have the same ionic strength of the respective ion. By comparing sedimentation rates in these solutions, an additional criterion of the binding properties of the glycocalyx can be obtained. It is herein termed ESSK, and it is defined by the ratio of the sedimentation rate in a solution containing a sodium salt to the rate in a solution containing a potassium salt. In typical embodiments the ESSK is the ratio of the sedimentation rate in a solution containing Na⁺ over the sedimentation rate in a solution K⁺, where the concentration of sodium and potassium are selected in the range from about 100 mM to about 150 mM. In some embodiments the concentration of sodium and potassium are selected in the range from about 110 mM to about 140 mM. The concentration of sodium and potassium may for example be about 115 mM or about 135 mM. In one embodiment the ESSK is the ratio of the sedimentation rate in a solution containing 125 mM Na⁺ over the sedimentation rate in a solution containing 125 mM K.

Where for erythrocytes from a subject a large ratio of sedimentation in a sodium salt solution vs. potassium salt solution is determined, this indicates high selectivity of specific Na⁺ binding, when compared to K⁺, to the negatively charged glycocalyx. If a low ratio of sedimentation in a sodium salt solution vs. potassium salt solution is determined, this indicates low selectivity of specific Na⁺ binding to the glycocalyx, when compared to K⁺. Since chronic Na⁺ overload is thought to damage the endothelial glycocalyx (reflected by the respective erythrocyte zeta potential), an increased value of ESSK indicates a strong Na⁺ affinity when compared to K⁺, and thus a poor glycocalyx. Accordingly, if a large ratio of sedimentation in a sodium salt solution vs. potassium salt solution is detected, it can be assessed that the respective subject is sensitive to sodium intake and at an increased risk to develop hypertension.

In some embodiments both the ESS and the ESSK are being assessed. As explained explained above, the ESS is the ratio of erythrocyte sedimentation velocities in higher over lower sodium salt concentration, for instance 150 mM Na⁺ over 125 mM Na⁺. It can be taken to define a measure for the Na⁺ sensitivity of the glycocalyx. The ESSK is the ratio of erythrocyte sedimentation velocities in sodium over potassium salt concentration, for instance 125 mM Na⁺ over 125 mM K. It can be taken to define a measure for the Na⁺ selectivity of the glycocalyx. A high ESS indicates a low Na⁺ binding capacity of the glycocalyx and vice versa. A high ESSK indicates a high affinity for Na⁺ binding of the glycocalyx and vice versa. Low-capacity/high-affinity for Na⁺ can be taken to characterize a poor glycocalyx, a high-capacity/low-affinity to characterize a well-developed glycocalyx.

In embodiments where both the ESS and the ESSK are being assessed, as an overall parameter for the quality/quantity of the glycocalyx both ratios can be multiplied with each other. This term is herein also referred to as OSS (overall sodium sensitivity). If the above exemplary concentrations of 150 mM Na⁺ over 125 mM Na⁺, as well as 125 mM Na⁺ over 125 mM K⁺ are used, the OSS derives from:

OSS=ESS×ESSK

OSS=(150 mM Na⁺/125 mM Na⁺)×(125 mM Na⁺/125 mM K⁺)

In this equation 125 mM Na⁺ can be canceled. Then, OSS consists only of two remaining determinants to be measured. It reads as follows:

OSS=150 mM Na⁺/125 mM K⁺

It is understood that instead of the exemplary 150 mM sodium and the 125 mM potassium other values may be applicable, according to the solutions selected. However, the above simplified equation only applies if the lower sodium concentration of the ratio of erythrocyte sedimentation velocities in higher over lower sodium salt concentration equals the concentration used for the ratio of erythrocyte sedimentation velocities in sodium over potassium salt concentration. Accordingly, the lower salt concentration used for determining ESS should be the same as the salt concentration used for determining ESSK. In conclusion, OSS is based only on two sedimentation rates which further simplifies the experimental procedure.

A sodium chloride solution and/or a potassium chloride solution as used in a method disclosed herein may in some embodiments include for example one or more buffer compounds. Numerous buffer compounds are used in the art and may be used to carry out a method as described in this document. Examples of buffers include, but are not limited to, solutions of salts of phosphate, carbonate, succinate, carbonate, citrate, acetate, formate, barbiturate, oxalate, lactate, phthalate, maleate, cacodylate, borate, N-(2-acetamido)-2-amino-ethanesulfonate (also called (ACES), N-(2-hydroyethyl)-piperazine-N′-2-ethanesulfonic acid (also called HEPES), 4-(2-hydroxyethyl)-1-piperazine-propanesulfonic acid (also called HEPPS), piperazine-1,4-bis(2-ethanesulfonic acid) (also called PIPES), (2-[tris(hydroxylmethyl)-methylamino]-1-ethansulfonic acid (also called TES), 2-cyclohexyl-amino-ethansulfonic acid (also called CHES) and N-(2-acetamido)-iminodiacetate (also called ADA). Any counter ion may be used in these salts; ammonium, sodium, and potassium may serve as illustrative examples. Further examples of buffers include, but are not limited to, triethanolamine, diethanolamine, ethylamine, triethylamine, glycine, glycylglycine, histidine, tris(hydroxymethyl)aminomethane (also called TRIS), bis-(2-hydroxyethyl)-imino-tris(hy-droxymethyl)methane (also called BIS-TRIS), and N-[Tris(hydroxy-methyl)-methyl]-glycine (also called TRICINE), to name a few. In some embodiments the pH of one or both of the two sodium chloride solutions is adjusted to a certain value such as a value in the range from about 6.0 to about 8.0. If the pH value is being set to a certain value, in typical embodiments both sodium chloride solutions are set to the same value. In some embodiments the pH value of the sodium chloride solutions is adjusted to a value that at least essentially corresponds to the physiological pH value of arterial blood, i.e. a pH value of about 7.4. The pH value is considered as about physiological if it is within the range of 7.35 to 4.45.

In some embodiments one or both of the first and the second solution used, e.g. one or both of two sodium chloride solutions or a sodium chloride solution and a potassium chloride solution, further include a monosaccharide. Many monosaccharides are known in the art. In some embodiments the monosaccharide is a hexose. As illustrative examples glucose (also called dextrose, corn sugar or grape sugar), fructose (also called fruit sugar), galactose and mannose are named here as a hexose monosaccharide. In some embodiments a combination of any two or more hexose monosaccharides may be used in a method or use disclosed herein. In some embodiment the monosaccharide is a pentose. Examples of a pentose monosaccharide that may be included in a method or use include, but are not limited to, arabinose, ribose, and xylose, as well as combinations thereof. In some embodiments one or both of the two sodium chloride solutions further includes a disaccharide. Examples of a disaccharide include, but are not limited to, sucrose (saccharose), lactose and maltose, as well as combinations thereof. In some embodiments a so called sugar alcohol, i.e. a derivative of a monosaccharide or a disaccharide, which carries a further hydroxyl group instead of the formyl or keto group. Examples of a sugar alcohol include, but are not limited to, sorbitol, mannitol, xylitol, lactitol and maltitol.

The mono- or disaccharide or the sugar alcohol may be present in any desired concentration as long as the respective solution has a physiological osmolarity. The mono- or disaccharide may advantageously be used to adjust the osmolarity of the respective sodium chloride solution. The mono- or disaccharide may for example be present in the solution in a molar amount roughly in the range from about one third or about half, including about 0.65-fold of the amount of sodium chloride to about 5-fold or 10-fold the amount of sodium chloride, including equal to the amount of sodium chloride. In some embodiments sodium chloride may be present in the sodium chloride solution in a molar amount roughly in the range from about one fifth of, including equal to the amount of the mono- or disaccharide to about three-fold or two-fold, including 1.5-fold of the amount of the mono- or disaccharide.

In typical embodiments one or both of the first and second solutions, e.g. two sodium chloride solutions or a sodium chloride solution and a potassium chloride solution further include a polymer such as a polysaccharide or a protein. In some embodiments the polymer has at least essentially no overall net charge, that is in a sodium chloride solution or a potassium chloride solutions used in a method disclosed herein or included in a kit disclosed herein the polymer is neutral if the respective sodium chloride solution is adjusted to physiological pH (supra). Of the many known polysaccharides typically a polysaccharide is selected that is soluble in water in the desired concentration. Examples of a suitable polysaccharide include, but are not limited to, glycogen, dextran, xanthan, and pectin, as well as combinations thereof. In typical embodiments the polymer, such as the polysaccharide or the protein, is present in both sodium chloride solutions in the same concentration. An illustrative example of a suitable protein is fibrinogen.

In some embodiments the polymer is dextran with an average molecular weight of about 40,000 Dalton or more. In some embodiments 80% or more of the dextran in the respective sodium chloride solution(s) has an average molecular weight of about 40,000 Dalton or more. As an illustrative example, 90% or more of the dextran in the respective sodium chloride solution(s) may have an average molecular weight of about 40,000 Dalton or more. The dextran included in a sodium chloride solution used in a method or included in a kit described in this document may have any desired molecular weight distribution and be from any desired source. In some embodiments the dextran has a molecular weight distribution that spans over 10,000 Dalton or more, including over 15,000 Dalton or more. In some embodiments the dextran has a molecular weight distribution in a range of about 5,000 Dalton or less, including about 2,500 Dalton or less. In some embodiments the dextran has a molecular weight distribution in a range of about 1,000 Dalton or less. In some embodiments the dextran has a molecular weight distribution in a range of about 2,000 Dalton or less. In some embodiments the dextran has a molecular weight distribution in a range of about 1,000 Dalton or less. In some embodiments at least essentially all of the dextran molecules in a sodium chloride solution present have a molecular weight of at least about 40,000 Dalton. In some embodiments at least essentially all of the dextran molecules in a sodium chloride solution present have a molecular weight of at least about 100,000 Dalton.

In some embodiments the polymer is dextran with an average molecular weight of about 70,000 Dalton or more. In some embodiments 70% or more of the dextran in the respective sodium chloride solution(s) has an average molecular weight of about 70,000 Dalton or more. As an illustrative example, 80% or more, including about 90% or more, of the dextran in the respective sodium chloride solution(s) may have an average molecular weight of about 70,000 Dalton or more. In some embodiments at least essentially all of the dextran molecules in a sodium chloride solution present in an embodiment disclosed herein have a molecular weight of at least 70,000 Dalton. In one embodiment a sodium chloride solution used/present has a molecular weight of about 70,000 Da. In some embodiments a sodium chloride solution used/present has an average molecular weight of about 200,000 Dalton to about 500,000 Dalton. If dextran is used as the polymer, it is typically included in a sodium chloride solution in an amount in the range from about 1% to about 10% (w/w), including in the range from about 1.5% to about 6% (w/w). In one embodiment the amount of dextran in a respective sodium chloride solution is selected in the range from about 2% to about 5%. As an illustrative example, the amount of dextran in a sodium chloride solution may be about 4% (w/w). As a further example, the amount of dextran in a sodium chloride solution may be about 3% (w/w).

In some embodiments the polymer is present in a sodium chloride solution in an amount that is selected in the range from about 0.01 to about 2 mmol/l. The range of the polymer may in some embodiments be from about 0.05 to about 1 mmol/l. As an example, the polymer may be included in a sodium chloride solution in a concentration of about 0.2 mmol/l. The polymer may for instance be a dextran, present in a concentration of about 0.2 mmol/l.

The polymer is thought to assist the aggregation of erythrocytes. Without being bound by theory it is assumed that the presence of polymer molecules in the sodium chloride solution has an inducing effect on erythrocyte aggregation once the erythrocytes have come in close proximity to one another. It has previously been suggested that a mechanism of depletion contributes to this assisting effect on aggregation albeit a bridging model has also been discussed.

In a method described herein erythrocytes from the subject are being suspended in a first and in a second solution as defined above. Generally about the same amount of erythrocytes per volume is being suspended in both solutions. Any desired container may be used for suspending the erythrocytes. If the erythrocytes have been obtained by centrifugation, the pellet that includes, essentially consists of, or consists of the erythrocytes may be resuspended in the respective sodium chloride or potassium chloride solution. Generally the suspended erythrocytes are placed in a container that allows distinguishing a clear supernatant from a suspension of erythrocytes. In this container the erythrocytes are being allowed to settle for a limited period of time (see below). The container generally has a circumferential wall, which may be a lateral wall, with a wall portion that allows light to enter the container, i.e. a light incident wall portion, and a light emerging wall portion, which is a wall portion that allows a view into the container to an extent that a clear supernatant and a cell suspension can be distinguished. At least one of these wall portions may for instance be a straight wall. In some embodiments these two wall portions are of the same material and thus both allow distinguishing a supernatant from a cell suspension. Examples of suitable material for the light incident wall portion and the light emerging wall portion include, but are not limited to, glass, quartz and plastic material. Suitable plastic materials for the light incident wall portion and the light emerging wall portion include, but are not limited to, polymethylmeacrylates (e.g. polymethyl-methacrylate (PMMA) or carbazole based methacrylates and dimethacrylates), polystyrene, polycarbonate, and polycyclic olefins. A further illustrative example of a material that is additionally suitable for a wall portion that allows light to pass only to a certain extent is fluoro-ethylen-propylen (FEP). In some embodiments the light incident wall portion and the light emerging wall portion are transparent or at least essentially transparent in the range of visible light. In one embodiment the light incident wall portion and the light emerging wall portion are at least essentially transparent. The transmission properties of a respective wall portion may also gradually or step-wise change from transparent to opaque, for example from one end of a respective wall portion to another end.

In some embodiments a suspension of erythrocytes is prepared in the same container in which the erythrocytes are afterwards being allowed to settle. In some embodiments a suspension of erythrocytes is prepared in a container that differs from the container in which the erythrocytes are being allowed to settle subsequently. The suspension of erythrocytes is in such embodiments transferred to the container in which the erythrocytes are being allowed to settle. In some embodiments the container into which erythrocytes are being transferred is a tube, which may have a straight circumferential wall. A respective tube may for instance be a capillary, which may have a sealable end. Such a capillary may have an open end and a sealed end.

Further, the erythrocytes in the suspension in the sodium chloride solution(s) and/or potassium chloride solution(s) are being allowed to settle for a limited period of time, i.e. they are allowed to settle for a period of time in which a suspension of erythrocytes and a supernatant form. Put differently, the period of time selected for allowing the erythrocytes to settle is short enough to avoid the formation of a pellet of cells at the bottom of the respective container. The period of time for allowing the erythrocytes to settle, which may also be addressed as allowing the erythrocytes to sediment or allowing the erythrocytes to associate with each other, may in some embodiments be selected in a range from about 20 minutes to about 240 minutes. The erythrocytes may for example be allowed to settle for a period of time from about 30 minutes to about 200 minutes. The period of time is in some embodiments about 45 minutes. In some embodiments the erythrocytes are allowed to settle for about 80 minutes. As an illustrative example, the erythrocytes may have been suspended in two saline solutions of different sodium chloride concentration, which encompass dextran and which have an approximately physiological osmolarity, i.e. about 260 mOsm/L. The suspensions may then be left unagitated for about 80 minutes, thereby allowing the erythrocytes to settle. In some embodiments the erythrocytes may be allowed to settle for about 135 or for about 150 minutes.

As noted above, while the erythrocytes are allowed to settle, the erythrocytes aggregate, i.e. the density of erythrocytes in the solution increases. As a result a supernatant forms—that is, a layer of the sodium chloride or potassium chloride solution above a suspension of erythrocytes which is a layer free of erythrocytes. While the erythrocytes continue to aggregate, the size of the supernatant increases. Since the rate of aggregation differs between the suspension with the high sodium chloride concentration and the suspension with the low sodium chloride concentration, a difference in the height of the supernatants forming as a top layer on the respective two solutions can be observed. This difference is allowed to increase at least until a clear and significant difference can be detected. Often a duration of about 20 minutes is sufficient to clearly distinguish the height of the supernatants between the two suspensions. In some embodiments a predetermined time interval is observed for allowing the two suspension of erythrocytes to settle. In some embodiments the two suspension of erythrocytes are allowed to remain unagitated for a period from about 25 minutes to about two hours, such as from about 35 minutes to about 100 minutes.

In some embodiments a method disclosed herein includes carrying out a control measurement. The control measurement is in some embodiments a measurement that may be carried out using erythrocytes from the same subject that were collected at a different point in time, for example one or more months or one or more years before the current measurement is being carried out. In some embodiments the control measurement is carried out with erythrocytes from another subject. Such other subject may be a subject with a blood pressure of known salt sensitivity and/or a patient of a known risk level of occurrence of hypertension. Salt sensitivity and/or risk level of occurrence of hypertension may for instance be average for a respective age group. The difference in height of supernatant between the two suspensions with erythrocytes from the subject may be compared to the difference in height of supernatant observed in the control measurement. In some embodiments the difference in height of supernatant between the two suspensions is converted into a ratio. Such a ratio may for instance be the height of supernatant between the first and the second solution, or put differently, the change in height of erythrocyte suspension (below the supernatant) of one of the first and the second solution over the change in height of erythrocyte suspension of the other solution. In some embodiments a ratio is determined of the height of supernatant of the second solution over the height of supernatant of the first solution.

In some embodiments the difference in height of the supernatants is compared to a predetermined reference value. In some embodiments the reference value is based on the difference between height of supernatants of erythrocytes of a control sample. A control sample may for example be a blood sample or a blood cell sample of a subject known not to be at elevated risk of suffering from hypertension or from aspects of a hypertension induced disease. In some embodiments a reference value is based on a control or reference value obtained concomitantly with the value of the sample from the subject. In some embodiments a respective control or reference value is determined at a different point in time, for example at a point in time earlier than the measurement of the sample from the subject is carried out. It is understood that the terms ‘control’ and ‘reference’ may in some embodiments be a range of values.

In some embodiments the determined difference in height of supernatants is regarded as increased relative to the difference in height of a reference value where the obtained value is about 1.2 times or more higher, including about 1.5 times, about two fold, about 2.5-fold, about three fold, about 3.5 fold, about 4-fold, about 5-fold or more higher than the expression level determined in a control sample. In some embodiments the determined difference in height is regarded as higher than a control where the obtained value is increased by an amount that is about 0.8-fold or more of the difference in height detected in a control sample. The determined difference in height may for example be regarded as different from the difference in height of a control if a value is about 30%, such as about 40% or about 50% higher or more than the difference in height determined in a control sample. In some embodiments a difference in height is regarded as different if the obtained value is about 60%, including about 80% higher than the difference in height in a control sample. A difference in height of supernatants is in some embodiments regarded as different if the obtained value is higher by about 20%, such as about 25% or more when compared to the difference in height between supernatants of a control sample.

A reference value may serve as a basis for a threshold value. If the difference in height of the supernatants between a first and a second solution exceeds the threshold value, the subject may be diagnosed as being at an increased risk of developing hypertension and/or of developing a hypertension induced condition.

Population studies may also be used to select a threshold value. Receiver Operating Characteristic (“ROC”) arose from the field of signal detection theory developed during World War II for the analysis of radar images, and ROC analysis is often used to select a threshold able to best distinguish a diseased subpopulation from a nondiseased subpopulation. A false positive in this case occurs when a person tests positive, but actually does not have the disease. A false negative, on the other hand, occurs when the person tests negative, suggesting the person is healthy, when it actually does have the disease. To draw a ROC curve, the true positive rate (TPR) and false positive rate (FPR) are determined as the decision threshold is varied continuously. Since TPR is equivalent with sensitivity and FPR is equal to 1—specificity, the ROC graph is sometimes called the sensitivity vs (1—specificity) plot. A perfect test will have an area under the ROC curve of 1.0; a random test will have an area of 0.5. A threshold is selected to provide an acceptable level of specificity and sensitivity.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

The comparison to a threshold value, which may be a predetermined threshold value, can be carried out manually, semi-automatically or in a fully automated manner. In some embodiments the comparison may be computer assisted. A computer assisted comparison may employ values stored in a database as a reference for comparing an obtained value or a determined amount, for example via a computer implemented algorithm. Likewise, the comparison to a reference measurement may be carried out manually, semi-automatically or in a fully automated manner, including in a computer assisted manner. A computer assisted comparison may rely on the storage of data, for instance in connection with determining a threshold value, on the use of computer readable media. Suitable computer readable media may include volatile, e.g. RAM, and/or non-volatile, e.g. ROM and/or disk, memory, carrier waves and transmission media such as copper wire, coaxial cable, fibre optic media. Exemplary carrier waves may take the form of electrical, electromagnetic or optical signals conveying digital data streams along a local network or a publically accessible network such as the Internet.

In some embodiments a method as described above is repeated after a period of one or more days, such as after one or more weeks, including one or more months, such as four, six, eight months or more. The repeated performances of the method may be carried out independent from one another. Hence, time intervals or combinations used may be independently selected. In some embodiments the repeated performances of the method may be carried out with exactly identical settings. For individual instances of measurements of repeated performances of a method described herein the composition of the first and the second solution may be independently selected. In some embodiments the measurements of formation of a supernatant of an erythrocyte suspension are carried out using the same composition of two sodium chloride solutions or of a sodium chloride solution and a potassium chloride in the repeated instances of carrying out the method or use.

In some embodiments where a plurality of sessions of a method disclosed herein are being carried out, in each session a plurality of measurements of supernatant height differences are been carried out. In some of these embodiments with repeated supernatant height detection these repeated measurements are taken within a time interval of less than a day such as less than three, or less than one hour.

In some embodiments a plurality of sessions of a method described herein is carried out in order to monitor a subject's risk of developing hypertension. The measurement of supernatant height differences may in such embodiments be repeated after a period of several months such as about 6 or about 9 months or after a period of several years such as about 2 years, about 5 years or about 10 years. One session may in such a monitoring serve as a reference session. This session may be carried out in the same way, i.e. using a pair of two sodium chloride solutions or of a sodium chloride solution and a potassium chloride with the same composition as in another session. A respective reference session may be a session where a method described herein is being carried out for the first time.

In some embodiments a method or use disclosed herein is a method or a use for diagnosis or for stratifying the risk of disease. The term “risk stratification” as used herein generally includes identifying subjects having a prognosis of developing hypertension, subjects having a prognosis of exacerbation in hypertension, as well as identifying subjects having a low, a medium and a high risk of developing hypertension. The term includes finding subjects having a blood pressure that is sensitive to sodium intake. The term includes finding healthy subjects whose blood pressure may rise to hypertension levels in the future. The term also includes finding subjects suffering from hypertension and whose blood pressure is sensitive to sodium intake. In this regard a method disclosed herein may be useful in both diagnosis and treatment. Measures may for example be taken to reduce the subject's sodium amount in the body, for example to increase sodium clearance and/or to reduce sodium intake. Stratification may be based on the probability (or risk) of developing or of exacerbation of hypertension. A method or use disclosed herein may also serve in stratifying the probability of the risk of any given cardiovascular disease or the risk of any given cardiac or cardiovascular event for a subject.

A large variety of hypertension related disorders are known in the art. Hypertension is known to result in damage and/or disorders of various organs, in particular of the heart, the brain, the kidney and the retina of the eye. In some embodiments a hypertension related condition is a cardiovascular disease. A risk of a condition to which a method or use according to the invention relates (supra) includes, but is not limited to, a risk of atherosclerosis, of coronary atherosclerosis, of vascular stenosis, of thrombosis, of peripheral vascular disease, including peripheral artery disease, of stroke, of haemorrhagic stroke, of ischemic stroke, of ischemic heart disease, of congestive heart failure, of Angina pectoris, myocardial infarction of ventricular arrhythmias, of myocardial ischemia, of coronary heart diseases, of acute coronary syndrome, of (acute) myocardial infarct, of cardiac insufficiency, of angina pectoris, of renal artery stenosis or of renal nephrosclerosis. “Ischemic heart disease” or myocardial ischemia as used herein is understood as referring to a disease characterized by reduced blood supply to the heart muscle, usually due to coronary artery disease (atherosclerosis of the coronary arteries). A “stenosis” as used herein, is defined as an abnormal narrowing in a blood vessel or other tubular organ or structure. “Cardiac insufficiency” as used herein refers to as an acute or chronic inability of the heart to supply sufficient blood to the tissue, and as a result, oxygen, to guarantee tissue metabolism at rest and under stress. Clinically, cardiac insufficiency is present, when typical symptoms (dyspnea, fatigue, liquidity retention) exist, the cause of which is based on a cardiac dysfunction within the meaning of a systolic or diastolic dysfunction.

In some embodiments where an increased difference in height between the supernatants is detected, the subject's blood pressure is determined. It may be decided to determine the subject's blood pressure at certain time intervals if the difference in height between the supernatants is found to be elevated. The subject's blood pressure may be monitored, for instance on an annual, on a monthly or on a weekly basis. Blood pressure may be determined according to any desired method at any one or more arteries of a subject. In typical embodiments peripheral blood pressure is determined, for example at one or more limbs of the subject. In some embodiments arterial blood force, i.e. the pressure applied by blood to the arterial wall, is determined. In some embodiments a blood pressure measurement of a human may for instance be carried out in the form of the indirect method first described by von Riva Rocci Recklinghaus. In this method an inflatable cuff is placed on the middle third of the upper arm and the pressure within the cuff is quickly raised up to complete cessation of circulation below the cuff, typically by inflating the cuff to a predetermined level. An electronic blood pressure measuring device or a reservoir of mercury at the end of a vertical glass column, in combination with listening to the artery just below the cuff with a stethoscope, may be used to determine the blood pressure value. In some embodiments an automatic, typically non-invasive, blood pressure gauge based on e.g. a pressure sensor, for example in combination with a press mechanism and/or a flow sensor and a sphygmomanometer, may be used.

A respective blood pressure measurement may be repeated. Such a measurement may be a measurement of the systolic blood pressure, a measurement of the diastolic blood pressure or both. Where a plurality of blood pressure measurements is carried out, the blood pressure measurements may be taken, at least substantially, after unitary time intervals or after varying time intervals. In some embodiments repeated determination of the subject's blood pressure is carried out at intervals of a certain, e.g. predetermined, length. As an illustrative example, the time interval between repeated measurements of the blood pressure may in some embodiments be selected in the range from about 6 hours to about 12 months, such as from about a day to about 6 months or from about a week to about 2 months.

A kit as described herein may include, including consist of, a pair of tubes and a container that includes a sodium chloride solution. The sodium chloride solution may further include a mono- or disaccharide such as glucose as well as a polysaccharide. In some embodiments the kit includes two containers that include a sodium chloride solution. Typically both containers with sodium chloride solution include at least essentially the same components with the only difference being the concentration of sodium chloride. In some embodiments the kit includes two containers, one container including a sodium chloride solution, and one container including a potassium chloride solution. Typically both containers with sodium chloride solution or with a sodium chloride solution and a potassium chloride solution include at least essentially the same components with the only difference being the concentration of sodium chloride or the presence of potassium chloride instead of sodium chloride. The kit may be used for carrying out a method as described above. The kit may be for prognosis, diagnosis and/or risk stratification of disease. The first container may be used for a session of the method described herein that serves as a control measurement.

Additional objects, advantages, and features of this disclosure will become apparent to those skilled in the art upon examination of the following examples thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art, and that such modifications and variations are considered to be within the scope of this disclosure.

### EXAMPLES

The following example serves in illustrating an embodiment of carrying out a method as disclosed herein. Furthermore it is illustrated how the interaction of erythrocytes (red blood cells; RBC) and vascular endothelium can be assessed using atomic force microscopy (AFM).

I: Salt-Blood-Test (SBT)

A. Protocol of carrying out the method based on venous puncture
1. 1 ml of blood (for double-measurement) is drawn by venous puncture using EDTA-K or heparinized monovettes (Sarstedt Company, Sarstedt, Germany).
2. Blood is transferred into 2 ml plastic vials (Eppendorf AG, Hamburg, Germany) and centrifuged for 5 minutes at 1800×g.
3. Plasma and buffy coat are removed and erythrocytes washed 2 times in 30 ml of HEPES-buffered (10 mM HEPES=2-(4-(2-Hydroxyethyl)-1-piperazinyl)-ethane sulfonic acid) sodium chloride (150 mM NaCl) including 1% bovine serum albumin (PAA Clone, Coelbe, Germany).
4. 80 μl of washed erythrocytes (ERY) are suspended in 120 μl NaCl solution (150 mM NaCl, Roth Company, Karlsruhe) containing 3% dextrane (Sigma 44886, MW: 70,000 Dalton).
5. 50 mM sucrose (Sigma 0389) is added to low NaCl solutions (125 mM) for maintaining constant osmolality (in relation to the 150 mM NaCl).
6. Hematocrit capillary tubes (Safecap P75-2000; length: 75 mm; Scholz Company, Neubiberg, Germany) are filled by capillary forces with the respective erythrocyte solutions (150 mM NaCl and 125 mM NaCl, as selected).
7. The hematocrit capillary tubes, closed on the lower end, are put on stands in an upright position.
8. Sedimentation rates of red blood cells are measured between 60 and 120 minutes and ESS (Erythrocyte Salt Sensitivity) is calculated (from the 75 minutes) values as follows:

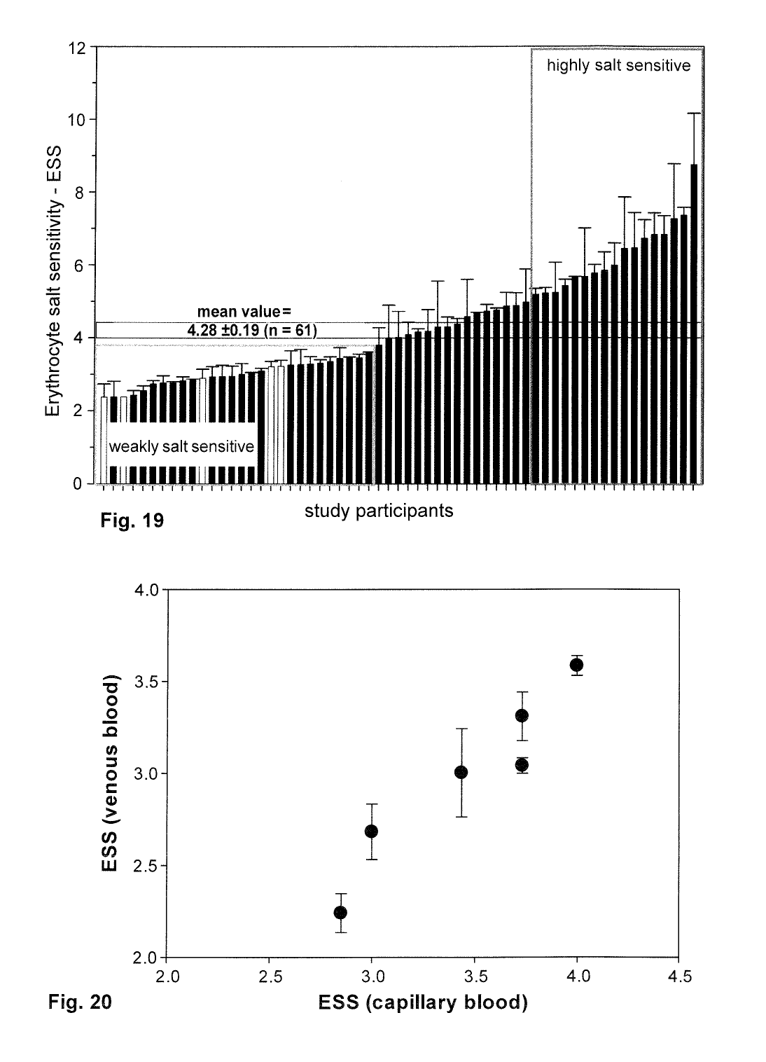

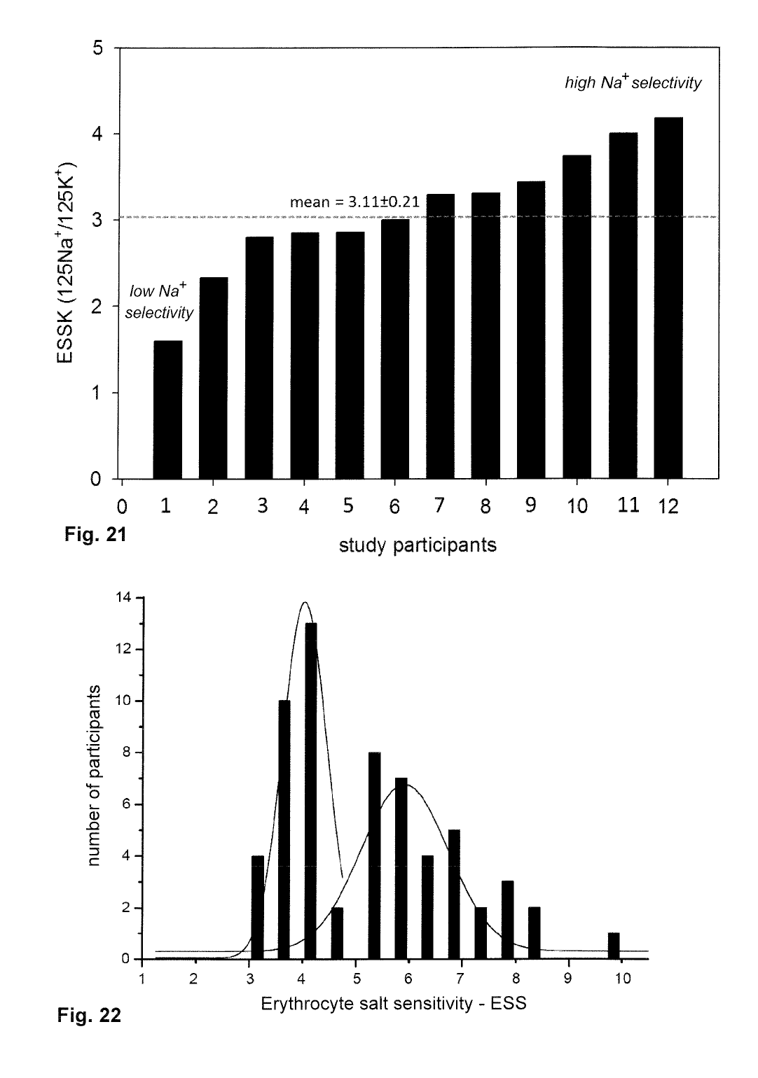

ESS=L₁₅₀/L₁₂₅ (as used in FIGS. 19, 20, 22)
or expressed as a percentage:

ESS (%)=[1−(L₄₂₅/L₁₅₀)]×100 (as used in FIGS. 5,6)

B: Protocol of carrying out the method based on capillary blood from finger tip
1. 200 μl of capillary blood is taken by puncture of the fingertip using hematocrit capillaries (3.75 IU Na-heparin/capillary; Hirschmann Laborgeräte, Eberstadt, Germany).
2. Blood is transferred into 2 ml plastic vials (Eppendorf AG, Hamburg, Germany) and centrifuged for 5 minutes at 1800×g.
3. Plasma and buffy coat are removed and erythrocytes washed twice in 1400 μl of HEPES-buffered (10 mM HEPES=2-(4-(2-Hydroxyethyl)-1-piperazinyl)-ethane sulfonic acid) sodium chloride (150 mM NaCl) including 1% bovine serum albumin (PAA Clone, Coelbe, Germany).
4. In order to obtain an in vitro hematocrit of 40%, 35 μl of washed erythrocytes are suspended in 52.5 μl NaCl solutions (125 and 150 mM NaCl, respectively; Carl Roth Company, Karlsruhe, Germany; each solution contains 3% dextrane; Sigma 4486, MW: 70,000) and mixed for 30 sec on a shaker (750 rounds per minute).
5. 50 mM sucrose (Sigma 0389) is added to low NaCl solutions (125 mM) for maintaining constant osmolality (in relation to the 150 mM NaCl).
6. Hematocrit capillary tubes (Safecap P75-2000; length: 75 mm; Scholz Company, Neubiberg, Germany) are filled by capillary forces with the two respective erythrocyte solutions (as described above).
7. The hematocrit capillary tubes, closed on the lower end, are put on stands in an upright position.
8. Sedimentation rates are measured between 60 and 120 minutes and ESS (Erythrocyte Salt Sensitivity) is calculated (from the 75 minutes) values as follows:

ESS=L₁₅₀/L₁₂₅ (as used in FIGS. 19, 20, and 22)

ESS can also be expressed as a percentage, calculated (from the 75 minutes) as:

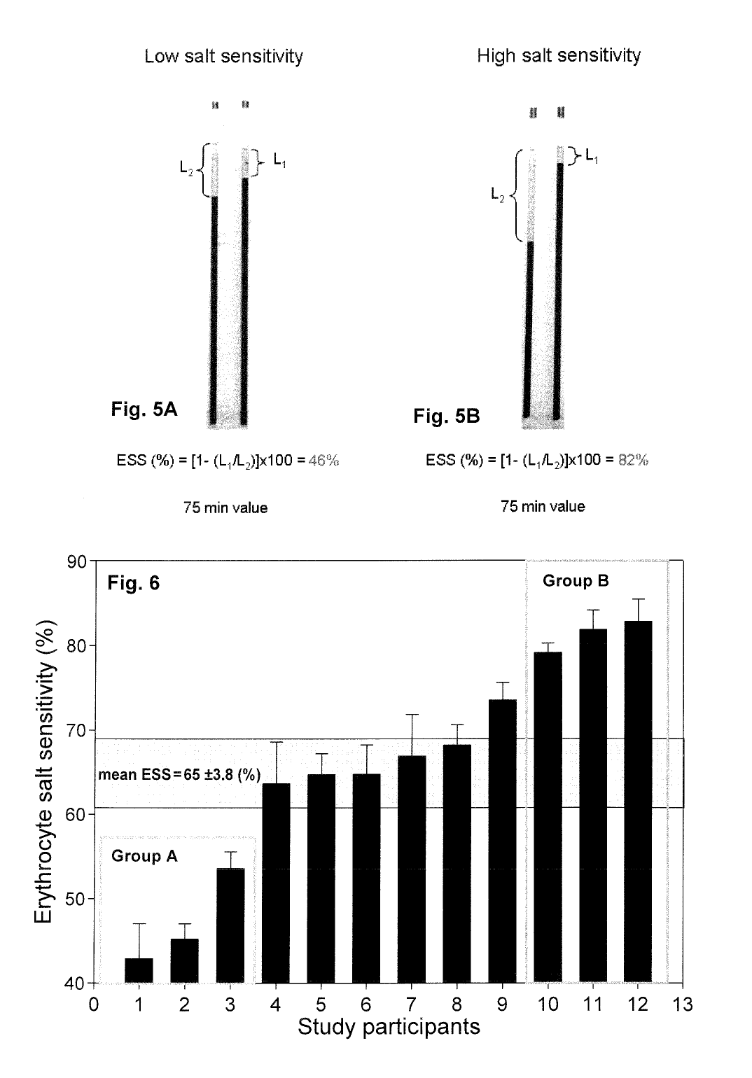

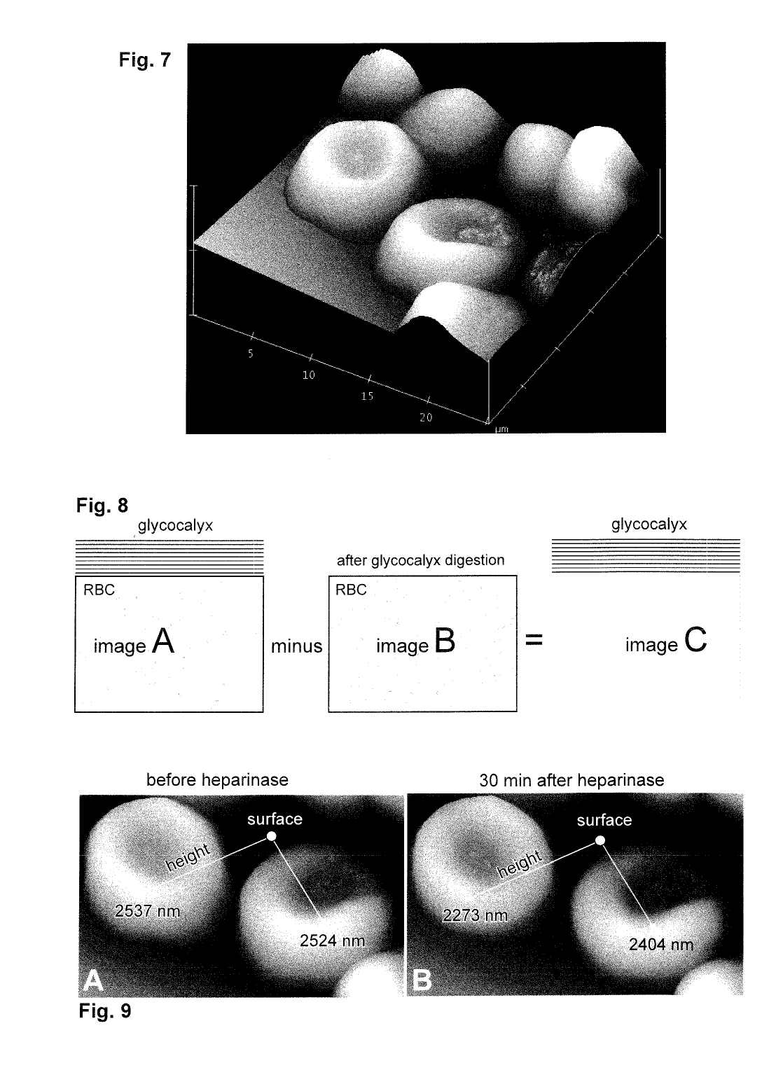

ESS (%)=[1−(L₁₂₅/L₁₅₀)]×100 (as used in FIG. 5 and FIG. 6)

The test was carried out on blood samples from 12 test persons (cf. FIG. 6).

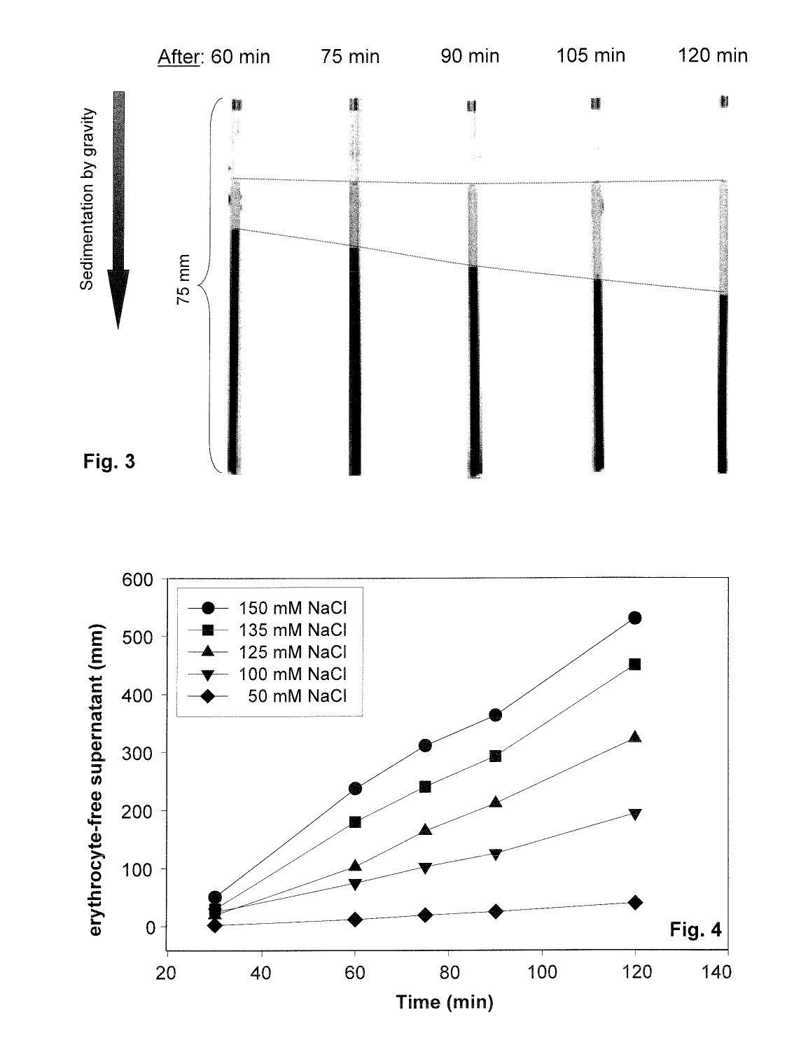

In contrast to the standard erythrocyte sedimentation rate (ESR), which is performed in whole blood and strongly dependent on any inflammation-relevant proteins in the plasma phase, the sedimentation rate used for ESS measurements is independent of any proteins, but strongly dependents on the NaCl concentration of the electrolyte solution in which the erythrocytes have been suspended. In particular, sedimentation rate depends on the RBC zeta potential and, thus, on the red blood cell glycocalyx. Without being bound by theory, since Na⁺ neutralizes the negative charges of the red blood cell surface, it can be assumed that sedimentation rate reflects the Na⁺ buffer capacity of the red blood cell glycocalyx. Dextran is typically to be added to all solutions for aggregation to occur. In pilot studies, the inventors found that a particular suitable molecular mass of the dextran is 70,000 D, and an advantageous concentration is 3%. FIG. 3 shows the time dependence of the red blood cell sedimentation rate. Initial sedimentation rates are quite linear, but they saturate with progression of time. This is due to the fact that a a hematocrit of 0.40 was used so that after extended time periods (hours), virtually all red blood cells, not only the large red blood cell aggregates, concentrate in the lower part of the tube. Therefore, the 60-min values were chosen for analysis. FIG. 4 shows the dependence of the erythrocyte sedimentation on NaCl concentration. Ion strength, and in particular the cation Na⁺, determines sedimentation velocity.

FIG. 5 displays two representative measurements. From the respective supernatants, ESS could be calculated. The ESS of FIG. 5A was 2.2, indicating that RBC sedimentation rate was 2.2 times larger in 150 mM Na⁺ than the sedimentation rate in 125 mM Na⁺. The ESS of FIG. 5B was 5.8, indicating that RBC sedimentation rate was 5.8 times larger in 150 mM Na⁺ than that in 125 mM Na⁺. Thus, ESS of blood used in FIG. 5B was more than twice as high as the ESS of blood used in FIG. 5A. In a series of control experiments, the heparan sulfate residues of the RBC glycocalyx were enzymatically removed by heparinase I incubation (Oberleithner, H., Pflügers Arch. (2013) doi:10.1007/s00424-013-1288-y), and then, ESS was measured.

In six experiments, ESS increased only by about 13% (from 2.6±0.12 to 3.0±0.14), indicating that not only heparin sulfate residues but also other negatively charged (and heparinase-insensitive) components of the glycocalyx contribute to the sodium-dependent sedimentation rate. FIG. 22 depicts ESS measured in 61 healthy volunteers of similar age. The frequency distribution indicates two ESS peaks, one at about 3 and another one at about 5. FIG. 19 shows these data in more detail. Forty-six percent (28 out of 61) of the study participants exhibited an ESS of at least 20% below average. Twenty-eight percent (17 out of 61) of the study participants exhibited an ESS of at least 20% above average. No significant gender difference of the respective ESS was observed. It is worth mentioning that the salt blood test has been performed in all experiments at a fixed hematocrit (0.40). Taken together, the data indicate that there is a wide range of ESS (from 2 to 8) within the normal population. It should be mentioned that at least five individuals (white bars in FIG. 19) were on low-salt diets (for no obvious reason, they indicated this explicitly in a questionnaire). Remarkably, the ESS of all five of them was found in the “weakly salt-sensitive” group.

II: Salt-Blood-Test by Measuring the Na+ Over K+ Ratio (ESSK)

Protocol based on capillary blood from the fingertip (excluding a centrifugation step)
1. 200 μl of capillary blood is taken by puncture of the fingertip using hematocrit capillaries (3.75 IU Na-heparin/capillary; Hirschmann Laborgeräte, Eberstadt, Germany).
2. Blood is transferred into a 500 μl plastic vial (Eppendorf AG, Hamburg, Germany) and Erythrocytes allowed to sediment for 90 minutes by gravidy at room temperature.
3. Plasma and buffy coat are removed and the erythrocyte concentrate (about 100 μl) at the bottom of the plastic vial taken up by a pipette and then 40 μl of erythrocyte concentrate pipetted into two (empty) 500 μl vials (40 μl each).
4. In order to obtain an in vitro hematocrit of 40%, 60 μl of a NaCl or a KCl solution (125 mM; each solution contains 3% dextrane; Sigma 4486, MW: 70,000) was added and mixed.
5. 50 mM sucrose (Sigma 0389) has been added before for maintaining normal osmolality (about 300 mosmol/l).
6. Hematocrit capillary tubes (Safecap P75-2000; length: 75 mm; Scholz Company, Neubiberg, Germany) are filled by capillary forces with the two respective erythrocyte solutions (as described above).
7. The hematocrit capillary tubes, closed on the lower end, are put on stands in an upright position.
8. Sedimentation rates are measured between 60 and 120 minutes and ESS (Erythrocyte Salt Sensitivity) is calculated (from the 60 minutes) values as follows:

ESSK=L_{125 Na}⁺/L_{125 K}⁺

III: Analysis of the Interaction of Erythrocytes and Vascular Endothelium Using Atomic Force Microscopy

Protocol of Carrying Out the Method:
1. Endothelial monolayers (Eahy629 cell line [Oberleithner, H., et al., Proc Natl Acad Sci USA (2007) 104, 16281-16286; Oberleithner, H., et al., Proc Natl Acad Sci U S A (2009) 106, 2829-2834] cultured on the bottom of 75 cm² culture flasks) are incubated at 37° C. with whole blood (5 ml) taken from volunteers over a time period of 18 to 22 hours. The flasks are continuously agitated (1 to 5 RPM, 7 degree angle) so that the RBC can physically interact with the surface of the endothelial cell layer.
2. The experiment is performed on intact endothelium and on endothelium that has been pretreated with heparinase (heparinase I, Sigma Aldrich H 2519, Taufkirchen, Germany) in order to remove the negatively charged heparan sulphate residues from the endothelial glycocalyx.
3. After this procedure the RBC are harvested, seeded on poly-l-lysine coated glass cover slips, fixed with 0.1% fixative (glutaraldehyde in HEPES buffer) and imaged by using an atomic force microscope.

AFM Imaging:

Imaging of the RBC surface was performed with methods basically described previously [Oberleithner, H., et al.: Nanoarchitecture of plasma membrane visualized with atomic force microscopy. Methods in Pharmacology: Ion channel localization methods and protocols. Edited by: A. Lopatin and C. G. Nichols. 2001. Humana Press Inc.; Oberleithner, H., et al., J Membr Biol (2003) 196, 163-172; Oberleithner, H., et al., Hypertension (2004) 43, 952-956; Schneider, S. W., et al., Methods Mol Biol (2004) 242, 255-279]. In order to image the glycocalyx of RBC the following protocol was used: A glass coverslip (diameter 15 mm), covered with RBC was mounted on the stage of the AFM and individual RBC were imaged in buffered electrolyte solution. Then, 2 U/ml of heparinase were added to the RBC. The RBC surface was continuously imaged over 30 minutes while the enzyme was active. Images were stored on the computer. After the experiment the respective images before and after application of heparinase were electronically substracted. This results in a so-called net-image that shows the glycocalyx (i.e. the enzyme-sensitive part of the RBC surface). Since the AFM generates 3-D images, height and volume of the images can be quantitatively evaluated. This complex and highly sophisticated method that needs in-depth AFM expertise and that is based on expensive AFM technology cannot be used for any clinical study.

The contents of the articles, patents, and patent applications, and all other documents and electronically available information mentioned or cited herein, are hereby incorporated by reference in their entirety to the same extent as if each individual publication was specifically and individually indicated to be incorporated by reference. In case of conflict, the present specification, including definitions, will control. Applicants reserve the right to physically incorporate into this application any and all materials and information from any such articles, patents, patent applications, or other physical and electronic documents.

The listing or discussion of a previously published document in this specification should not necessarily be taken as an acknowledgement that the document is part of the state of the art or is common general knowledge.

One skilled in the art would readily appreciate that the present methods, kits and uses are well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The methods, uses and kits illustratively described herein may suitably be practiced and applied in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereon It is recognized that various modifications are possible. Thus, it should be understood that although the present disclosure has been specifically disclosed by exemplary embodiments and optional features, modification and variation of the disclosures embodied therein herein disclosed may be resorted to by those skilled in the art, and that such modifications and variations are considered to be within the scope of this disclosure.

The invention has been described broadly and generically herein. Each of the narrower species and subgeneric groupings falling within the generic disclosure also form part of the methods, uses and kits. This includes the generic description of the methods, uses and kits with a proviso or negative limitation removing any subject matter from the genus, regardless of whether or not the excised material is specifically recited herein.

Other embodiments are set forth within the following claims. In addition, where features or aspects of the invention are described in terms of Markush groups, those skilled in the art will recognize that the invention is also thereby described in terms of any individual member or subgroup of members of the Markush group.

1. An in vitro method of analyzing whether the blood pressure of a subject is sensitive to sodium intake, the method comprising:
(a) suspending erythrocytes of the subject in one or more pairs of a first solution of about physiological osmolality and a second solution of about physiological osmolality, each first solution and each second solution comprising at least one of sodium chloride and potassium chloride,
wherein for each pair of first and second solution the first solution comprises sodium chloride, and the second solution comprises sodium chloride or potassium chloride,
wherein a second solution comprising sodium chloride has a sodium chloride concentration that is at least about 25 mM higher than the sodium chloride concentration of the first solution, and
wherein a second solution comprising potassium chloride has a concentration of potassium chloride that is of at least essentially about the same value as the concentration of sodium chloride in the first solution;
(b) allowing the suspended erythrocytes in the first and in the second solution of at least one pair of first and second solution to settle for a period of time sufficient to allow the formation of a supernatant; and
(c) detecting the difference in height of the supernatant between the first and the second solution of the at least one pair of first and second solution,
wherein an increased difference in height of the supernatants of the at least one pair of first and second solution, relative to a threshold value, indicates that the blood pressure of the subject is sensitive to sodium intake.

2. The method of claim 1, wherein the method is a method of assessing the susceptibility of a subject to develop hypertension, and wherein an increased difference in height of the supernatants relative to a threshold value, indicates an increased susceptibility of the subject to develop hypertension.

3. The method of claim 1, wherein each of the first and the second solution of a pair of a first solution and a second solution has a concentration of sodium chloride or potassium chloride that is about 100 mM or more.

4. The method of claim 1, wherein for one or more pairs of a first and a second solution of about physiological osmolality, a second solution comprising potassium chloride has a concentration of potassium chloride that is about 25 mM or more higher than the sodium chloride concentration of the first solution.

5. The method of claim 1, wherein the first and the second solution of a pair of a first solution and a second solution further comprise a polysaccharide.

6. The method of claim 5, wherein the polysaccharide is dextran.

7. The method of claim 5, wherein the polysaccharide has an average molecular weight of about 70,000 Da.

8. The method of claim 5, wherein the polysaccharide is comprised in the first and the second solution in an amount of about 3%.

9. The method of claim 1, wherein at least one of the first and the second solution of a pair of a first solution and a second solution comprises a monosaccharide or a disaccharide.

10. The method of claim 9, wherein the disaccharide is sucrose.

11. The method of claim 1, wherein the threshold value is based on the difference in height of the supernatant between a corresponding first and a corresponding second solution of erythrocytes of a control sample.

12. The method of claim 1, wherein the period of time for allowing the suspended erythrocytes to settle is selected in the range from about 45 to about 120 minutes.

13. The method of claim 1, comprising suspending erythrocytes of the subject in a first and a second pair of solutions, each pair of solutions comprising a first solution and a second solution,
wherein for the first pair of solutions, both the first solution and the second solution comprise sodium chloride, and
for the second pair of solutions the first solution comprises sodium chloride, and the second solution comprises potassium chloride, and
wherein the concentration of sodium chloride in the first solution of the first pair of solutions is at least about the same as the concentration of potassium chloride in the second solution of the second pair of solutions.

14. A kit of parts comprising a pair of tubes and one or more pairs of a first and a second container, a first container comprising a first solution and a second container comprising a second solution, each first solution and each second solution comprising at least one of sodium chloride and potassium chloride, wherein for each pair of a first and a second container the first solution comprises sodium chloride, and the second solution comprises sodium chloride or potassium chloride, wherein a second solution comprising sodium chloride has a sodium chloride concentration that is higher than the sodium chloride concentration of the first solution, and wherein a second solution comprising potassium chloride has a concentration of potassium chloride that is of at least about the same value as the concentration of sodium chloride in the first solution.

15. The kit of claim 14, wherein for a pair of a first and a second container, a second solution comprising sodium chloride has a sodium chloride concentration that is at least about 25 mM higher than the sodium chloride concentration of the first solution.

16. The kit of claim 14, wherein each of the first and the second solution is of about physiological osmolality.

17. The kit of claim 14, wherein each of the first and the second solution has a concentration of sodium chloride or potassium chloride of about 100 mM or more.

18. The kit of claim 14, wherein each of the first and the second solution further comprises a polysaccharide.

19. The kit of claim 14, wherein for a pair of a first and a second container at least one of the first and the second solution comprises a monosaccharide or a disaccharide.

20. The kit of claim 14, wherein each of the tubes of the pair of tubes is a capillary, the capillary having an open end and a sealable end.
